(19) United States
(12) Patent Application Publication
(10) Pub. No.: US 2025/0127412 A1
(43) Pub. Date: Apr. 24, 2025

(54) ASSESSING MICROCIRCULATION

(71) Applicant: ODI MEDICAL AS, Oslo (NO)

(72) Inventors: Anne Kari KVERNEBO, Oslo (NO); Knut KVERNEBO, Oslo (NO); Svein-Erik MÅSØY, Oslo (NO)

(21) Appl. No.: 18/681,371

(22) PCT Filed: Oct. 21, 2021
(86) PCT No.: PCT/EP2021/079284

§ 371 Date: Feb. 5, 2024

(30) Foreign Application Priority Data
Aug. 5, 2021 (GB) 2111314.7

(51) Int. Cl. A61B 5/026; A61B 5/00; A61B 5/02; A61B 5/0205; A61B 5/1455; A61B 90/20

(52) U.S. Cl. CPC A61B 5/0261; A61B 5/0042; A61B 5/0205; A61B 5/4836; A61B 5/4848; A61B 5/02007; A61B 5/14551; A61B 90/20; A61B 2576/026

(57) ABSTRACT

The present invention relates to a method of identifying or monitoring circulatory failure in a subject, which method comprises assessing the microcirculation in the limbus and optionally also the bulbar conjunctiva of the subject, said method comprising assessing the following parameter(s) in the subject's limbus and optionally also in their bulbar conjunctiva: (i) functional capillary density (FCD); and/or (ii) capillary flow velocity (CFV); wherein parameters (i) and (ii) are assessed by microscopy. The present invention also relates to apparatus and software designed for performance of such a method.

Microcirculation is the circulation of the blood in the microvasculature present within organ tissues. The present invention relates to the analysis of the microcirculation in the limbus and bulbar conjunctiva of a subject, and in particular to methods of, and apparatus for, such analysis and to the use of data obtained thereby. For example, data obtained by means of the present invention may be used to diagnose or assess the prognosis of subjects. The methods of the invention can also provide an early warning of circulatory problems prior to a clinical diagnosis.

Circulatory failure can be defined as the inability of the cardiovascular system to supply sufficient amounts of oxygen to meet the metabolic demands of the cells of the body. Since the circulation of blood refers to its continual flow from the left side of the heart, through branching arteries, to reach and traverse the microscopic vessels in all parts of the body, returning via the veins to the right side of the heart, to flow on through the lungs and back to the left heart again, disease processes in the heart, the lungs and the transport vessels, as well as in the microcirculation, can cause circulatory failure.

These conditions may develop acutely or over time. Lack of oxygen delivery may lead to cellular dysfunction or death, and may proceed to organ failure and death of an individual. Circulatory failure can be local or systemic. Generalized (i.e. systemic) and clinically evident failure, i.e. shock, may be central (e.g. caused by heart failure or hypervolemia) or peripheral (e.g. distributive failure caused by sepsis).

As well as systemic circulatory failure, which may threaten life, there may be localised circulatory failure (which may itself be life threatening, for example, if the organ affected is essential or if the affected region could become necrosed and result in sepsis) such as erythromelalgia. Even if not life threatening, circulatory failure may lead to physical impairment, chronic health conditions and reduced quality of life. No reliable and accepted parameter or set of parameters has been established to assess the microcirculation and make clinical decisions on the data generated.

Clinical examination of arterial and venous circulation may give valuable information, but conclusions are often wrongly extrapolated to be valid for conclusions of microvascular function. A large number of technologies, like blood gas analyses and assessment of metabolic products in blood samples, pressure and cardiac output measurements, as well as imaging techniques, are used to diagnose and guide treatment of circulatory failure. These techniques collect data assessing function of the heart, veins and arteries, as well an average index of the metabolic function of the body. However, the same problem applies to these measurements as to clinical assessments: measured values within the reference spectrum can coexist with critical systemic or local circulatory failure.

A myriad of different parameters and measuring techniques for diagnosing and assessing circulatory failure exist, as mentioned above. These may include blood tests, for example to determine acid-base balance in arterial blood or levels of lactate in serum. Arterial and venous circulation may be measured: here techniques include imaging using contrast media (angiography, venography and magnetic resonance (MR) assessments); Doppler ultrasound measurements of blood flow velocities; and invasive and non-invasive blood pressure measurements.

The main functions of conjunctival vessels are to transport and deliver oxygen and nutrients for metabolic needs, serve thermoregulation of the eye and serve a “watch out” function in case of infection or inflammation.

As described in WO2014/114814, the present inventors have previously developed a method of recording, analysing and evaluating microcirculatory parameters using video microscopy and spectroscopy. WO2014/114814 describes assessments performed on the microcirculation of the skin in order to determine circulatory failure. Reference is also made to possible investigation of the tongue or conjunctiva but no such assessments are reported. The conjunctiva is treated as a single region and there is no reference to the bulbar conjunctiva in particular or to the neighbouring limbus.

The present inventors have subsequently identified that measuring certain microcirculatory parameters specifically in the limbus and/or bulbar conjunctiva region of the conjunctiva is of particular diagnostic value, and for the first time have developed a method of analysing limbus microcirculatory parameters. The inventors have, for the first time, assessed oxygen delivery from the capillaries to the cells of the conjunctiva and established that the different functions of the two regions of the conjunctiva (limbus and bulbar conjunctiva) mean that they behave differently under circulatory stress. They have shown that relevant clinical information can be obtained by looking at these specific regions and more especially by comparing the parameters of interest across the two regions. The inventors have determined that assessing particular microcirculatory parameters specifically in these regions provides advantages in the assessment, diagnosis and monitoring of local eye conditions, neurological conditions and systemic circulatory failures.

Thus, in one aspect, the present invention provides a method of identifying or monitoring circulatory failure in a subject, which method comprises assessing the microcirculation in the limbus and optionally also the bulbar conjunctiva of the subject, said method comprising assessing the following parameter(s) in the subject's limbus and optionally also in their bulbar conjunctiva:
(i) functional capillary density (FCD); and/or
(ii) capillary flow velocity (CFV);
wherein parameters (i) and (ii) are assessed by microscopy.

In some embodiments, the microscope uses white, unpolarised light.

In some embodiments, the assessment is performed on data previously acquired from the subject and the subject is not still undergoing monitoring.

In another aspect, the present invention provides a method of making a prognosis for a subject with circulatory failure which method comprises assessing the microcirculation in the limbus and optionally also the bulbar conjunctiva of the subject, said method comprising assessing the following parameter(s) in the subject's limbus and optionally also in their bulbar conjunctiva:
(i) functional capillary density (FCD); and/or
(ii) capillary flow velocity (CFV);
wherein parameters (i) and (ii) are assessed by microscopy.

In some embodiments, the microscope uses white, unpolarised light.

In some embodiments, the assessment is performed on data previously acquired from the subject and the subject is not still undergoing monitoring.

In preferred embodiments, parameters (i) and (ii) are both assessed. In preferred embodiments the limbus and the bulbar conjunctiva are both assessed. In some particularly preferred embodiments, parameters (i) and (ii) are both assessed in both the limbus and the bulbar conjunctiva. The present inventors have surprisingly found that these parameters and these regions, alone or considered together, can give useful clinical information about the subject, in particular whether and to what extent they are suffering from circulatory failure.

Thus, in a preferred embodiment, the invention provides a method of identifying or monitoring circulatory failure in a subject, which method comprises assessing the microcirculation in the limbus and the bulbar conjunctiva of the subject, said method comprising assessing the following parameter(s) in the subject's limbus and bulbar conjunctiva:
(i) functional capillary density (FCD); and/or
(ii) capillary flow velocity (CFV);
wherein parameters (i) and (ii) are assessed by microscopy; and optionally
comparing measurements obtained in respect of parameters (i) and/or (ii) in the subject's limbus with measurements obtained in respect of the same parameter(s) in the subject's bulbar conjunctiva. Preferably parameters (i) and (ii) are assessed.

In a further preferred embodiment, the invention provides a method of identifying or monitoring circulatory failure in a subject, which method comprises assessing the microcirculation in the limbus and optionally also the bulbar conjunctiva of the subject, said method comprising assessing the following parameters in the subject's limbus and optionally also in their bulbar conjunctiva:
(i) functional capillary density (FCD); and
(ii) capillary flow velocity (CFV);
wherein parameters (i) and (ii) are assessed by microscopy; and optionally
comparing measurements obtained in respect of parameter (i) with measurements obtained in respect of parameter (ii). Preferably both the limbus and bulbar conjunctiva (BC) are assessed.

In another aspect, the invention provides a method of identifying or monitoring circulatory failure in a subject, or making a prognosis for a subject, or providing clinically relevant information about a subject, or monitoring the efficacy of treatment in a subject,
which method comprises
processing, implemented by a computer, of image(s) of the microcirculation in the limbus and optionally also the bulbar conjunctiva of the subject previously acquired using a microscope, to obtain value(s) for the following parameter(s) in the subject's limbus and optionally also in their bulbar conjunctiva:
(i) functional capillary density (FCD); and/or
(ii) capillary flow velocity (CFV).

In embodiments, the method of the invention further comprises
processing, implemented by a computer, of image(s) of the microcirculation in the bulbar conjunctiva of the subject, to obtain value(s) for the following parameter(s) in the subject's bulbar conjunctiva:
(i) functional capillary density (FCD); and/or
(ii) capillary flow velocity (CFV);
wherein parameters (i) and (ii) are assessed by microscopy.

In embodiments, value(s) for one or more of the following parameters are also obtained:
(a) heterogeneity of the FCD;
(b) heterogeneity of CFV;
(c) oxygen saturation of microvascular erythrocytes (SmvO₂); and
(d) heterogeneity of SmvO₂;
In embodiments, the value(s) of parameters (a) and (b) are generated using the image(s) and the value(s) of parameters (c) and (d) are generated using data previously acquired by diffuse reflectance spectroscopy (DRS).

In embodiments, value(s) for 2 or more, more preferably 3 or 4 of parameters (a) to (d) are obtained, most preferably in both the limbus and bulbar conjunctiva.

Optionally the method of the invention comprises, before the step of processing by the computer of the image(s) and optionally the data (i.e. the DRS data), receiving by the computer of the image(s) and optionally the data (i.e. the DRS data).

The present inventors have observed for the first time a compensatory mechanism between features of the microcirculation in the conjunctiva (including the limbus) when the body is under stress, e.g. experiencing circulatory failure, whether local or systemic. Adequate delivery of oxygen to the tissues is dependent on a number of factors, in particular the distribution of capillaries and the flow of oxygenated blood through those capillaries. Deviation in FCD or CFV (or more especially in both parameters) from normal, healthy values, is a sign that the body is causing the microcirculation to compensate for some degree of circulatory failure. Thus, FCD may be at normal or higher than normal levels, while CFV is below (perhaps significantly below) normal levels. On the other hand, CFV may be at normal or higher than normal levels, while FCD is below (perhaps significantly below) normal levels. In both scenarios oxygen delivery is reduced and circulatory failure exists.

Intuitively one might consider that a greater capillary density or flow velocity is always preferred but this has been shown not necessarily to be the case. For example, the inventors have observed that an over-transfused patient on ECMO displayed supra-normal values for FCD, but this was combined with sub-normal CFV and the net effect was reduced oxygen delivery. On the other hand, a sub-normal FCD may reduce oxygen delivery in spite of optimal CFV, because of increased oxygen diffusion distances.

Likewise, a supra-normal CFV has been shown to be a sign of dysregulation of microvascular function because, while many erythrocytes pass through each capillary, the capillary transit time is low leading to less extraction of oxygen from each erythrocyte. With sub-normal CFV few erythrocytes pass though the capillary, and although the capillary transit time is prolonged and gives plenty of time for oxygen delivery from each erythrocyte, the total delivery capacity is reduced because few erythrocytes pass though the capillary.

As shown in the Examples, when a continuous flow pump applied to sedated pigs is used as a model for impaired circulation, these two key parameters individually (or preferably when considered together) show deviation between healthy and sick subjects and enable diagnosis or prognosis of circulatory failure. It should be noted that even those pigs receiving pulsatile flow (which is more supportive of normal circulation than continuous flow) also, over time, give insight into the effects on the microcirculation during circulatory failure, as the test set up still compromises the animal's circulation as compared to a fully healthy animal.

Thus, in preferred embodiments, the study of CFV and FCD in regions of the eye provides a surprisingly good and simple to perform indication of circulatory failure. These two parameters may be assessed in the limbus, preferably also in the BC. The assessment of both parameters, typically in comparison with healthy reference values and/or over time, gives a unique window into circulatory health.

Without wishing to be bound by theory, it is believed that the physiological relationship between the limbus and BC makes these two regions particularly sensitive to local or systemic circulatory failure and parameters (i) and (ii) particularly informative. The limbus is the more metabolically active region and the BC can be considered to provide a transport function of oxygen to the limbus. When the body's circulation is challenged, e.g. due to a disease process or injury, CFV in the limbus (optionally also or alternatively in the BC) may be reduced; it has surprisingly been found that the FCD in the BC increases as a compensatory mechanism. Thus these characteristics act as markers of circulatory failure.

Thus, when one of parameters (i) and (ii) is below normal and one is above normal, either within the limbus or the bulbar conjunctiva or across these two regions, circulatory failure is indicated. “Normal” means as observed in comparable healthy subjects. Circulatory failure may be indicated when only one parameter is significantly deviated from normal values.

Elevated FCD in the bulbar conjunctiva, either over time, as compared to reference values (e.g. in a healthy subject) or compared to the limbus, is indicative of circulatory failure or of more severe or worsening circulatory failure. Also, decreased CFV in the limbus and/or BC as compared to reference values (e.g. in a healthy subject), is indicative of circulatory failure or of more severe or worsening circulatory failure. When these two effects, i.e. elevated FCD in the bulbar conjunctiva and decreased CFV in the limbus or BC (especially when it is decreased in both the limbus and BC), are both seen, this is highly indicative of circulatory failure or of more severe or worsening circulatory failure.

Thus decreased CFV in the BC, either over time or as compared to reference values (e.g. in a healthy subject) is indicative of circulatory failure or of more severe or worsening circulatory failure, particularly when seen with elevated FCD in the bulbar conjunctiva (BC) as compared to reference values (e.g. in a healthy subject).

According to the present invention, “circulatory failure” (CF) is a failure to deliver oxygen in sufficient amounts to cells in a tissue (local CF) or in several tissues (systemic CF). According to this definition, CF is a microcirculatory failure. A sufficient amount of oxygen is that amount required meet the metabolic demands of the cells.

In some embodiments, one or more additional parameters may be assessed in either or both of the limbus and BC, in particular one or more of:
(a) heterogeneity of the FCD;
(b) heterogeneity of CFV;
(c) oxygen saturation of microvascular erythrocytes (SmvO₂); and
(d) heterogeneity of SmvO₂;
   may be assessed. Parameters (a) and (b) are assessed by microscopy and parameters (c) and (d) are preferably assessed by diffuse reflectance spectroscopy (DRS). Preferably 2 or more, more preferably 3 or 4 of parameters (a) to (d) are assessed, most preferably in both the limbus and BC.

Decreased SmvO₂ in the limbus or BC, either over time or as compared to reference values (e.g. in a healthy subject) is indicative of circulatory failure or of more severe or worsening circulatory failure, particularly when seen with increased FCD in the BC or decreased CFV in the bulbar conjunctiva or limbus.

The parameters are assessed in the limbus and bulbar conjunctiva using the same methodologies, i.e. using the same methodologies to obtain microscopy images and optionally DRS spectra, and the same downstream analysis tools. Preferably, the microscopy images (and where appropriate) DRS spectra from the two regions are or have been obtained from the subject in a single visit to the site of analysis, e.g. hospital, for instance preferably within 6 hours, more preferably within 3 hours, more preferably within 1 hour of each other.

Preferably, test results can be compared against a database of comparator values, i.e. from previously obtained values.

Any comparison between limbus and bulbar conjunctiva parameter measurements may be performed by a human user, or may be automated using computer software. The comparison of parameters in the limbus and bulbar conjunctiva may be a direct comparison of said values. Optionally, however, first deviations of said values from reference/control values obtained from the same conjunctival area are calculated, and then the extent of the deviations from reference/control values is compared between limbus and bulbar conjunctiva.

Preferably the parameter values obtained in the subject's limbus (and bulbar conjunctiva) are compared to reference values from the limbus (and bulbar conjunctiva) in a healthy reference subject (“healthy reference values”) and any deviations found.

Likewise, the parameter values obtained in the subject's limbus (and bulbar conjunctiva) may be compared to reference values from the limbus (and bulbar conjunctiva) in a pathology reference subject (“pathology reference values”) and any deviations found.

The limbus forms the border between the transparent, avascular cornea and the opaque, vascularized conjunctival tissues in the eye. The limbus is the region in which the avascular cornea and the vascularized conjunctiva fuse. It is defined for the purposes of the present invention as the vascular circumferential tissue adjacent to the cornea, it extends no more than 1 mm from the limbal/corneal boundary and the target region of interest for assessment according to the present invention is typically the region up to 0.6 or 0.5 mm from the limbal/corneal boundary. The limbus is located between the bulbar conjunctiva and the cornea.

The bulbar conjunctiva of the eye (also referred to herein as the bulbus) comprises of two or three layers of non-keratinized cuboidal stratified epithelium resting on a basement membrane with Goblet-and Langerhans cells embedded. Underlying the epithelium is a stroma that contains blood vessels, nerves, fibroblasts, melanocytes, Langerhans cells and immune competent cells. The bulbar conjunctiva (BC) target region of interest for assessment according to the present invention is at least 2 mm from the limbal/corneal boundary.

The limbal conjunctiva is the region of the conjunctiva found within the limbus. Throughout the application the term “limbus” and “limbal conjunctiva” are used interchangeably. The limbus may be considered anatomically separate from the conjunctiva or part thereof but, for convenience herein, it may be referred to as part of the conjunctiva.

The present methods concern assessment of microcirculation, so it is only the section of the limbus with vasculature, i.e. the limbal conjunctiva, which is of interest. Although the limbus also comprises corneal tissue, the corneal tissue is avascular, and so is not of relevance to the present invention. It is understood that assessment of the limbus or bulbar conjunctiva in the present methods is the assessment of the stroma layer therein, since it is the stroma that comprises blood vessels.

Microcirculation is the circulation of the blood in the microvasculature present within organ tissues. However, the term “microvasculature” is not defined consistently in the field. Under some definitions used, the term includes all blood vessels of ≤100 microns in diameter, and may include arterioles, capillaries, metarterioles, sinusoids and venules. However, in the methods of the present invention, the term “microvasculature” and “microvessels” is used to refer to capillaries, which are defined as those blood vessels≤20 microns in diameter.

In the methods of the present invention, preferably the assessments of the indicated parameters are repeated in different regions of the limbus/conjunctival area and the results summed or averaged.

The conjunctiva may be assessed inside the upper and lower eyelid.

The terms “assessing”, “measuring” and “analysing” are used interchangeably throughout, as are the terms “assessment”, “measurement” and “analysis”.

Assessment of the microcirculation may also comprise analysis of pericapillary pathology, such as determining whether pericapillary bleedings and/or dark haloes are visible.

In all methods of the invention, the subject may be a non-human animal or a human, but is preferably a human. The subject has or is suspected of having circulatory failure, circulatory failure may be localised or systemic.

The subject may be:
(i) being considered for or undergoing intensive care therapy, such as extra-corporeal membrane oxygenation (ECMO) and/or extra-corporeal life support treatment (ECLS); or
(ii) suffering from pre-eclampsia; or
(iii) suffering from sepsis; or
(iv) suffering from chronic or acute heart failure; or
(v) asphyxiated; or
(vi) suffering from acute or chronic respiratory failure; or
(vii) the recipient of an organ transplant.

The subject may be suffering from:
(i) An eye condition, preferably selected from glaucoma, wet age-related macular degeneration (wet AMD), pterygium, eye cancer, local inflammation, scleritis/episcleritis, stem cell failure, corneal transplant failure, keratoconus, ocular ischemic syndrome, Anterior Ischemic Optic Neuropathy (AION) or complication following cosmetic procedures performed on the eye or eye lids; or
(ii) A neurological condition, for example a circulatory disturbance of the brain such as ischemic or hemorrhagic stroke, multiple sclerosis, meningitis or increased intracranial pressure; or
(iii) Hypovolemic shock, diabetes with microangiopathy or prematurity.

In (i) the cancer may be for instance uveal melanoma, conjunctival melanoma or basal cell cancer of the eyelid. The local inflammation may be vasculitis, uveitis or due to allergy. The stem cell failure may be Fuchs dystrophia. The AION may be arteritic AION or non-arteritic AION, and may be due to for instance retinal artery embolus, retinal vein occlusion, chemical burns, or stroke.

Reference above and elsewhere herein to “suffering from” includes subjects suspected of suffering from one or more of the above conditions. Preferably the subject is presenting with one or more symptoms of one or more of the above conditions. Preferably the subject has been treated, or is currently undergoing treatment for one or more of the above conditions. The subject may have conjunctival pigmentation. Conjunctival pigmentation may be:
i) non-pathological pigmentation, for instance racial/ethnic melanosis, congenital melanocytosis, non-pathological hypermelanosis or Axenfeld nerve loop;
ii) naevus (congenital or acquired) or primary acquired melanosis, which may be non-pathological (i.e. without atypia) or may be pre-cancerous;
iii) pathological, for instance due to conjunctival melanoma, extension of a pigmented intraocular tumor to the conjunctival region, pathological hypermelanosis, conjunctival argyrosis, Bitot spot (vitamin A deficiency), sub-conjunctival haemorrage, Addison's disease or alcaptonuria;
iv) secondary conjunctival pigmentation, for instance caused by irradiation, hormonal changes, chemical irritation, or chronic inflammatory conjunctival disorders;
v) chemically induced, e.g. due to prescription drugs including but not limited to chlorpromazine and topical epinephrine;
vi) exogenous pigmentation, for instance tattooing or staining of the eye;
vii) due to foreign bodies in the conjunctiva for instance misapplied cosmetics (e.g. mascara).

Alternatively or in addition, the subject may have a dermoid cyst.

### Microscopy

Parameters (i) and (ii) (and (a) and (b)) are assessed by microscopy. The assessment comprises the use of a microscope, and preferably the use of a video microscope, to provide images, and/or the use of images (still and/or moving) obtained by (video) microscopy. The microscope is preferably digital and is preferably computer assisted video microscopy (CAVM).

The microscope preferably uses unpolarised light. The microscope preferably uses polychromatic light, e.g. white light, such as produced by a conventional microscope light source. White unpolarised light is preferred. A microscope “using” a particular type of light does not merely relate to the source of the light, but also to the nature of the light that contacts the subject/sample and the nature of the light that is used to generate the images. In other words, preferably, parameters (i) and (ii) and (a) and (b) are assessed by unpolarised white light microscopy.

Preferably all of parameters (i) and (ii) and (a) and (b) are assessed using the same instrument.

In one embodiment, the method comprises a user actively obtaining/gathering said microscopy images from a subject. Typically, the recording lens of the microscope/video microscope does not touch the conjunctiva of the subject; the method is non-invasive. A local anaesthetic such as oxibuprocaine may be applied to the eye prior to obtaining the images in order to reduce movement of the eye. Eyelid retraction, which is well-known in the field and is used routinely in examinations of the eye, may be performed if the user considers it necessary.

The anatomy of the eye is well documented and it is within the competencies of the person of ordinary skill in the art to obtain microscopy images of the desired conjunctival region, such as the limbus and/or the bulbar conjunctiva.

Preferably, the field of view of the microscope is less than or equal to 1.5 mm², preferably less than or equal to 1 mm².

Microscopy “images” may be single images, i.e. single frames or photographs. Preferably however, a video microscope is used, and the “images” may be video images, i.e. a “series” or “sequence” of images, i.e. a film such as a “CAVM film”. As used herein, the term “image(s)” is intended to refer expressly to both single images and series of images (films).

Films, such as CAVM films are preferably 5 to 25 seconds in length, preferably 10 to 20 seconds in length, e.g. about 15 seconds in length.

Preferably, the recording frame rate of the microscope (or CAVM or digital microscope) is 5 frames per second, preferably 10, 15, 20, 25 or 30 frames per second, more preferably 30 frames per second.

Preferably, the microscope is housed (or fixed) within (or together with) a slit lamp. This arrangement provides greater stability and control for the clinician when assessing the eye of the subject, and thus better precision.

Preferably multiple images are obtained from a single region of the conjunctiva. Preferably 2 to 10, more preferably about 5 images are obtained from a single region of the conjunctiva. Preferably the images are transmitted to a computer for storage. The gathering/obtaining of microscopy images may performed by a human or may be automated.

However, in a preferred embodiment, the microscopy images have been obtained previously and the method comprises analysis of the provided images. Thus, microscopy images may be analysed off-line, if necessary, remotely from the patient. Here “off-line” means without the microscope being in contact with or connected to the subject, e.g. after the completion of the gathering of the images. Thus in certain embodiments, the methods of the invention comprise assessing the subject's microcirculation through the analysis of microscopy images thereof. Again, microscopy images may be moving images (films) or still images (including still frames of moving images).

Optionally, the microscopy images are or have been transmitted to a computer for storage prior to assessment.

Analysis of images obtained by microscopy may comprise selecting one or more images based on pre-determined criteria (e.g. to ensure suitable quality), optionally adjusting/optimizing one or more image parameters for instance light intensity, contrast, sharpness or quality, and optionally drawing a series of parallel lines thereon to facilitate analysis (discussed further below). If the images are films, then analysis is preferably performed whilst the film is running, in case capillaries are visible in some but not all frames of the film. The selection, adjustment/optimization of images and the drawing of parallel lines thereon may be performed manually or automated via computer software.

Assessment of the microscopy images and assessment of the DRS spectra may be performed by a human, i.e. visually. Alternatively, the assessment(s) may be automated (i.e. performed by computer), whereby each image/spectra is scanned to identify the above-mentioned characteristics, e.g. using conventional recognition techniques. Automated assessment is also referred to as visual assessment herein. Thus, parameters (i) and (ii) and (a) and (b) are assessed visually. The values/numbers of the respective characteristics per unit area may then be calculated. In the case of CAVM, such processing may be carried out by the same computer to which the images were uploaded, or they may be transmitted to a computer for processing.

Optionally, analysis of the images and spectra will involve some computer processing and some analysis by individuals who are experienced in interpretation of the images/spectra.

Where pericapillary bleedings and/or dark haloes are present, the number per unit area and/or proportion of capillaries affected may be calculated.

### FCD

Preferably, FCD is defined as the number of capillary crossings per mm of “reference line” (or “grid line”).

Preferably, on a microscopy image, a plurality of parallel straight lines are drawn (i.e. superimposed, i.e. applied), either by a human user or automatically using computer software, within the conjunctival region of interest (e.g. in the limbus or in the bulbar conjunctiva). The parallel lines (“reference lines” or “grid lines”) are typically drawn on-screen using computer software. Preferably, the total number of parallel lines is 2 to 10, more preferably 4 to 8, more preferably 5 to 7, more preferably 6. The parallel lines are equally spaced. Preferably, the distance between the parallel lines is equivalent to 10-200 μm, more preferably 50 to 150 μm, more preferably about 100 μm. An image may be magnified on screen for ease of viewing, but the preferred spacing of parallel lines stated above is the preferred spacing in the conjunctival region itself, i.e. when the image of the conjunctival region has not been magnified, i.e. when the ratio of the apparent size of the conjunctival region to the actual size of the conjunctival region is 1:1.

Preferably, all of said parallel lines are drawn within the conjunctival region of interest. When drawing said lines within the limbus, it is important to first draw a line wherein the length of the line touches the edge of the cornea thereby defining the boundary of the cornea and the limbus. Thus, preferably, on a microscopy image of the limbus, a first straight line is drawn, either by a human user or automatically using computer software, wherein the length of the line touches the edge of the cornea, i.e. this first line defines the boundary of the cornea and the limbus. This line is referred to herein as the “limbus-cornea boundary line”. Subsequently, one or more further line(s) is/are drawn on the image within the limbus and parallel to the limbus-cornea boundary line. The lines are typically drawn on-screen using computer software. Preferably the lines are drawn on the image automatically by computer software. Preferably, the total number of parallel lines drawn is 2 to 10, more preferably 4 to 8, more preferably 5 to 7, more preferably 6. The parallel lines are equally spaced. Preferably, the distance between the parallel lines is equivalent to 10-200 μm, more preferably 50 to 150 μm, most preferably about 100 μm. An image may be magnified on screen for ease of viewing, but the preferred spacing of parallel lines stated above is the preferred spacing in the limbus itself, i.e. when the image of the limbus has not been magnified, i.e. when the ratio of the apparent size of the limbus to the actual size of the limbus is 1:1.

For the bulbar conjunctiva (BC) an analogous approach may result in 4 to 8, e.g. 6 horizontal and parallel lines being drawn within the BC. Preferably, the distance between the parallel lines is equivalent to 10-200 μm, more preferably 50 to 150 μm, most preferably about 100 μm. Unlike with the limbus, the drawing of the first line on the BC need not been drawn in any particular region of the BC, so long as all subsequent lines fall within the BC. Preferably, the lines on the BC are all drawn at least 2 mm away from the limbus-bulbar conjunctiva boundary.

When determining FCD, blood vessels of less than 20 microns in diameter are preferably counted as capillaries. Blood vessels larger than 20 microns are preferably not included in the analyses. The capillary density is “functional” in that the measured capillaries are observed to contain erythrocytes. Blood vessels not comprising erythrocytes are not included in the analysis; they are typically not visible in any case. On each of the drawn parallel lines including on the limbus-cornea boundary line, the number of instances of functional capillaries crossing said line is counted. Functional capillary density is then determined as the number of functional capillary crossings per mm of said lines. In other words, the six lines will have a total length in mm, and a total number of capillary crossings. These totals are used to determine the number of functional capillary crossings per mm of line in a given microscopy image.

The FCD in units of number of functional capillary crossings per mm of line can be determined manually by a human user or with the use of computer software. Preferably, it is determined automatically using computer software that identifies and counts said crossings.

FCD is preferably provided as a mean value, e.g. based on at least 4 repeated measurements, preferably 4 to 10 repeated measurements, for instance 5 repeated measurements. Thus according to the methods of the invention several separate images or video sequences are obtained and the FCD of each determined before a mean is calculated.

If video images are obtained, then each frame within the sequence of images is of the same conjunctival location, i.e. the microscope is not moved or repositioned whilst obtaining the images (recording).

When repeated measurements are made, i.e. when multiple images of the limbus or bulbar conjunctiva are obtained, whether still or video images, then the microscope is preferably moved randomly into a new position within the same region of interest (i.e. limbus or bulbar conjunctiva) after one image has been obtained and before the next image is obtained. This ensures that measurements are taken from images obtained at a variety of locations within the conjunctival region of interest. The FCD values in units of number of functional capillary crossings per mm of line from each image are then used to generate a mean value.

Heterogeneity of FCD is an indication of the variation seen between multiple values/measurements. In general, larger variations are a bad sign in terms of circulatory function and an increase in heterogeneity may be an early warning of circulatory failure and indicative of an over-stretching of the microcirculation's natural compensatory mechanisms for responding to challenges caused by disease. Compensatory mechanisms include opening of dormant capillaries, growth of new capillaries and changes in CFV.

The method for obtaining multiple FCD measurements within a conjunctival region of interest is described above.

Heterogeneity is preferably found by determining the coefficient of variance of measurements made from different locations in the same conjunctival area of interest (the co-efficient of variance—COV—is the standard deviation divided by the mean). These are most conveniently provided by analysing a plurality of images of randomly selected areas of the conjunctival region of interest, e.g. the limbus.

Heterogeneity preferably depends on analysis of at least 4 images, preferably 5-10 images, e.g. 5 images.

Line et al (1992) in Microvascular Research, 43, pp 285-293 describe (in the context of LD flowmetry measurements) how the number of samples required to provide a reliable mean and heterogeneity score may be derived.

Preferably heterogeneity of FCD is calculated automatically by computer software.

### Blood Capillary Flow Velocity (CFV)

CFV is, as the name suggests, the velocity of the flow of erythrocytes within a capillary. CFV may be measured for each of a plurality of microvessels. CFV analysis necessarily involves analysis of video images (films) that are running so that flow rates can be assessed.

It is sufficient for velocity to be estimated based on a number of categories. For example, there may be seven categories used, which may be assessed visually: 0=visible erythrocytes, no flow; 1=“sluggish flow” (intermittent slow flow); 2=“slow flow” (continuous slow flow or intermittent “no flow” and rapid flow); 3=continuous flow; 4=rapid flow (continuous rapid flow or intermittent slow and brisk flow); 5=brisk flow (continuous brisk flow); 6=uncertain (velocity impossible to determine).

Brisk flow relates to a flow rate significantly higher than normal which results in poor perfusion as the oxygen carried by the erythrocytes in such microvessels does not stay long enough in the microvessel to be delivered to the tissue. Such microvessels will act as a physiological arterio-venous shunt.

CFV is preferably determined by scoring each capillary identified within a film for velocity as described above. The mean CFV for a given film can then be determined as the sum of all values for velocities in the film divided by number of values.

Preferably, CFV is measured at each capillary crossing identified as described above in relation to the measurement of FCD. If the velocity is observed to fluctuate during a film sequence, then the average velocity throughout the film sequence is used.

Like FCD, CFV scoring can be determined manually by a human user or with the use of computer software. Preferably, it is determined automatically using computer software that identifies capillaries and determines the CFV category.

CFV is preferably provided as a mean value, e.g. based on at least 4 repeated measurements, preferably 4 to 10 repeated measurements, for instance 5 repeated measurements. Thus according to the methods of the invention several separate video sequences are obtained and the CFV of each is determined before a mean is calculated.

The microscope is not moved or repositioned whilst obtaining the images (recording).

When repeated measurements are made, i.e. when multiple videos of the limbus or bulbar conjunctiva are obtained, then the microscope is preferably moved randomly into a new position within the same region of interest (i.e. limbus or bulbar conjunctiva) after one video has been obtained and before the next video is obtained. This ensures that measurements are taken from videos obtained at a variety of locations within the conjunctival region of interest. The mean CFV values of each video are then used to generate an overall mean CFV value.

Preferably a mean categorical flow velocity is determined. The mean flow-categorical velocity (MFCV) may be determined for the capillaries on a given set of images, by the following formula:

MCFV={Fr(1)×1}+{Fr(2)×2}+{Fr(3)×3}+{Fr(4)×4}+{Fr(5)×5}

where Fr stands for the fraction of capillaries within each flow category. Such a calculation is described by Wester et al., (2011) Clinical physiology and functional imaging 31 (2): 151-158. In this calculation, capillaries in category 0 and 6 are excluded.

Alternatively, CFV may be provided as a median CFV value per image/film, and a mean of said median values determined based on repeated measurements as described above.

In practice, categories 0 and 3 are the predominant categories of vessels observed, and so, as an alternative to the MCFV value, capillary flow velocity may be assessed in terms of the number or proportion of capillaries categorised as 0 (negative sign when proportion is high) or the number or proportion categorised as 3 (positive when high).

Again, CFV or MCFV can be determined manually by a human user or using computer software. Preferably, it is determined automatically using computer software that identifies said crossings and scores flow velocities.

Heterogeneity of CFV is an indication of the variation seen between multiple values/measurements. In general, larger variations are a bad sign, as discussed above in relation to FCD.

The method for obtaining multiple CFV measurements within a conjunctival region of interest is described above.

Heterogeneity is preferably found by determining the coefficient of variance of measurements made from different locations in the same conjunctival area of interest (the co-efficient of variance—COV—is the standard deviation divided by the mean). These are most conveniently provided by analysing a plurality of images (films) of randomly selected areas of the conjunctival region of interest, e.g. the limbus.

Heterogeneity preferably depends on analysis of at least 4 films, preferably 5-10 films e.g. 5 films.

Line et al (1992) in Microvascular Research, 43, pp 285-293 describe (in the context of LD flowmetry measurements) how the number of samples required to provide a reliable mean and heterogeneity score may be derived.

Alternatively, heterogeneity of capillary flow velocity is preferably calculated using the formula:

{Fr(0)×3}+{Fr(1)×2}+{Fr(2)×1}+{Fr(3)×0}+{Fr(4)×1}+{Fr(5)×2}

where Fr stands for the fraction of capillaries within each flow category.

Preferably heterogeneity of CFV is calculated automatically by computer software.

### DRS and Oxygen Saturation

The present methods preferably further comprise measurement of oxygen saturation of microvascular erythrocytes (SmvO₂), and optionally heterogeneity thereof. The ability of the methods of the present invention (as reported in the Examples) to distinguish between the limbus and BC in respect of this parameter is highly advantageous. These parameters of microcirculation are assessed by diffuse reflectance spectroscopy (DRS). DRS spectra provide information about the amount of oxy- and deoxy-haemoglobin in the microvessels. This information can be used to estimate oxygen saturation of erythrocytes within the microvessels (SmvO₂).

The assessment comprises the use of a diffuse reflectance spectrometer to provide spectra, and/or the use of spectra obtained by DRS.

In one embodiment, the method comprises a user actively obtaining/gathering said spectra. The spectra are preferably obtained by applying the DRS probe (discussed below) gently to the conjunctiva of the eye (and/or other tissue). Preferably multiple spectra are obtained from different locations within the conjunctival region of interest, preferably at least 4 spectra, preferably 5-15 spectra, e.g. 10 or 12 spectra.

When repeated measurements are made, i.e. when multiple spectra are obtained, then the DRS probe is preferably moved randomly into a new position within the same region of interest (i.e. limbus or bulbar conjunctiva) after one spectra has been obtained and before the next spectra is obtained. This ensures that measurements are taken from spectra obtained at a variety of locations within the conjunctival region of interest. A mean value can then be obtained.

Preferably the spectra are transmitted to a computer for storage. The gathering/obtaining of spectra may be performed by a human or may be automated.

However, in a preferred embodiment, the spectra have been obtained previously and the method comprises analysis of the provided spectra. Thus, spectra may be analysed off-line, if necessary, remotely from the patient. Here “off-line” means without the DRS probe being in contact with or connected to the subject, e.g. after the completion of the gathering of the spectra. Thus in certain embodiments, the methods of the invention comprise assessing the subject's microcirculation through the analysis of spectra.

Optionally, the spectra are or have been transmitted to a computer for storage prior to assessment.

Analysis of spectra obtained by DRS may comprise selecting one or more spectra based on pre-determined criteria (e.g. to ensure suitable quality).

Assessment of the microscopy images and assessment of the DRS spectra may be performed by a human, i.e. visually. Alternatively, the assessment(s) may be automated (i.e. performed by computer), whereby each image/spectra is scanned to identify the above-mentioned characteristics, e.g. using conventional recognition techniques. The values/numbers of the respective characteristics per unit area may then be calculated. Such processing may be carried out by the same computer to which the images/spectra were uploaded, or they may be transmitted to a computer for processing.

DRS spectroscopy comprises applying an excitation/collection probe to the conjunctival area of the subject. In practice, this comprises initially placing the probe on the cornea (where there is no vasculature), which will result in a flat DRS curve being displayed on the screen of the probe, or to which the probe is connected. The user then moves the probe laterally whilst watching the screen until the characteristic double-bump-curve signal is displayed on screen. At this point, the probe is in direct contact with the conjunctiva and oxygen saturation data can be obtained from the precise area of interest.

The DRS probe comprises i) a light source, preferably a halogen light source connected to ii) one or more emitting optical fibres; and iii) one or more separate optical fibres for receiving reflected light from the subject, connected to iv) a spectrometer.

Oxy-haemoglobin and deoxy-haemoglobin result in characteristic and distinguishable DRS spectra peaks. DRS analysis of blood vessels results in a compound spectra due to the presence of both oxy-haemoglobin and deoxy-haemoglobin. Routine analysis of the spectra obtained from a DRS measurement of a subject's vasculature allows for the percentage oxygen saturation of the subject's blood to be determined.

SmvO₂ values may be presented as concentrations of oxyhemoglobin and deoxyhemoglobin (partial pressures of O₂) or preferably as “% saturation” of the blood in the region assessed.

Preferably, all investigated parameters are determined in the same area of the conjunctiva. In practice, the microscope is used to obtain image(s) of a conjunctival area, wherein the microscope is attached to an apparatus scaffold that is configured to permit replacement of said microscope with a DRS excitation/collection probe. After the microscopy image(s) have been obtained, the microscope is replaced within the scaffold with the DRS excitation/collection probe without movement of the subject or the scaffold. This results in the DRS probe being located in precisely the same position as the microscope, and allows the oxygen saturation to be determined in precisely the same conjunctival area from which the microscopy image(s) were obtained.

Preferably, the conjunctival area from which a DRS spectra is obtained is of an area within or equivalent to the field of view of the microscope used to obtain images for analysis of FCD and CFV, i.e. preferably an area less than or equal to 1.5 mm², preferably less than or equal to 1 mm². DRS probes are described in the literature as having a measuring volume (rather than a measuring area), however, the positioning of the DRS probe involves placing the probe on a conjunctival area of interest.

Standard DRS techniques typically involve measuring volumes that are much larger than 1 mm³, which provides information on SmvO₂ in the mixture of arterioles, capillaries and venules in the area assessed. In the methods of the present invention, the DRS measuring volume is ≤ 1 mm³, preferably about 0.1 mm³. Measurement of such a small volume in the conjunctiva ensures that the SmvO₂ measurements obtained are indicative of the SmvO₂ in the conjunctival capillaries in particular, which is desirable for assessing the subject's microcirculation. Although part of the signal may still come from arterioles or venules, rather than the capillaries, the contribution from arterioles or venules is minimized by use of the small DRS measuring volume.

In preferred embodiments, oxygen extraction by the microvessels is also determined; this is calculated as follows:
arterial oxygen saturation (SaO₂) minus SmvO₂

Arterial oxygen saturation is suitably measured using pulse oximetry, which is a well-known technique.

The methods of the invention also comprise an assessment of the heterogeneity of SmvO₂ and, where appropriate, heterogeneity of oxygen extraction. The heterogeneity of SmvO₂ is preferably found by determining the coefficient of variance of SmvO₂ over a plurality of different conjunctival locations, i.e. spatial heterogeneity.

Since each of the measuring volumes have a volume of fractions of 1 mm³, placing the probe on the conjunctiva, removing the probe and placing the probe on the conjunctiva again for a second assessment in the same area, is sufficient to obtain data from different measuring volumes.

Heterogeneity preferably depends on analysis of at least 4 spectra, preferably 5-15 images, e.g. 10 or 12 spectra.

Line et al (1992) in Microvascular Research, 43, pp 285-293 describe (in the context of LD flowmetry measurements) how the number of samples required to provide a reliable mean and heterogeneity score may be derived.

Preferably heterogeneity of SmvO₂ is calculated automatically by computer software.

Thus heterogeneity as determined according to parameters (a), (b) and (d) is an indication of the variation seen between multiple values. In general, larger variations are a bad sign. Heterogeneity is preferably found by determining the coefficient of variance over a plurality of different locations in the same area (the co-efficient of variance—COV—is the standard deviation divided by the mean). These are most conveniently provided by analysing a plurality of images/spectra of randomly selected areas of the conjunctival region of interest, e.g. the limbus. Heterogeneity preferably depends on analysis of at least 4 images/spectra, preferably 5-10 images/spectra, e.g. 6-8 images/spectra.

All assessments by microscopy and DRS may be performed in real time, i.e. with the patient present, alternatively, and in some cases preferably, the methods of the invention are performed on data obtained from the patient and the patient is not still undergoing monitoring or required to be present for the analysis to take place. This applies to all the methods of the invention.

All the methods of the invention may advantageously be repeated one or more hours or one or more days apart over several hours, days or weeks. For example an individual subject may be assessed more than 3, 5, 10 or 20 times and trend analysis performed to refine the diagnosis, prognosis or, in particular, assess the effectiveness of treatment.

When repeated measurements are performed it is possible to monitor disease progression. If measurements are taken before and after start of a new therapy, it is possible to quantify the effect of the new therapy for a patient (for example the effect of a drug or putting a patient on a heart-lung-machine (ECMO) in the case of acute heart failure). This may be a valuable tool in clinical medicine and can be used for “patient tailored therapy”. If the new therapeutic intervention does not lead to increased oxygen delivery (as determined by the methods of the invention), the intervention does not have the desired effect and may be stopped.

The output of the above analysis may comprise a separate measure for each of the characteristics (i), (ii) and optionally (a) to (d) that were determined. These may be displayed on a monitor associated with the computer, sent to a printer, etc. Alternatively, the outputs may be combined to provide one or more scores indicative of the pathology of the microcirculation. For example, a weighted sum or average of the individual characteristics may be determined and displayed as mentioned above. Preferably the comparison is made between a weighted sum or average of any 2 to 6 of the parameters, optionally all 6 of the parameters. An algorithm may be used to give a single output value based on a weighting applied to each parameter which may vary depending on clinical setting and patient characteristics.

### Diagnostic and Prognostic Uses

The present inventors have surprisingly shown that microscopy can be used to generate meaningful data from the limbus and BC of a subject regarding the health of the subject through analysis of their microcirculation. It was not expected that the limbus would be a particularly useful area for analysis, nor superior to other regions of the conjunctiva, nor that reproducible and reliable information about microvasculature/microcirculation could be obtained using non-invasive techniques. The limbus may be considered anatomically separate from the conjunctiva or part thereof but, for convenience herein, it may be referred to as part of the conjunctiva. Such information permits diagnostic and prognostic conclusions.

The inventors have recognised that the state of microcirculation in the limbus, optionally also together with the BC, provides valuable insight when diagnosing conditions and seeking to optimize therapy. It is believed that the identification/diagnosis of circulatory failure with the limbus can be made at an earlier stage than is the case today by other methods, and that this increase in sensitivity and the earlier recognition of circulatory failure, will result in earlier therapeutic interventions and better clinical outcomes than is achieved today.

Viewed from a still further aspect, the invention provides a method of providing clinically, e.g. diagnostically or prognostically, relevant information about a subject comprising assessing the microcirculation in the subject's limbus and optionally also in their bulbar conjunctiva in respect of the following parameters:
(i) functional capillary density (FCD); and/or
(ii) capillary flow velocity;
wherein parameters (i) and (ii) are assessed by microscopy.

The clinically relevant information is relevant to a disease state of the subject, i.e. whether or to what extent the subject is suffering from a disease or condition. It may also include information on the impact of a particular therapeutic intervention. The information is pertinent to the subject's circulatory health.

Relevant diseases or conditions are discussed above in the context of the subject who is assessed and include:
(i) An eye condition, preferably selected from glaucoma, wet age-related macular degeneration (wet AMD), pterygium, eye cancer, local inflammation, scleritis/episcleritis, stem cell failure, corneal transplant failure, keratoconus ocular ischemic syndrome or Anterior Ischemic Optic Neuropathy (AION); or
(ii) A neurological condition, preferably selected from stroke, multiple sclerosis or increased intracranial pressure; or
(iii) Systemic circulatory failure, preferably selected from chronic or acute heart failure, sepsis, hypovolemic shock, diabetes with microangiopathy and prematurity.

All diseases and conditions mentioned herein may be diagnosed or monitored using the methods of the invention. Thus, alternatively viewed, the present invention provides a method of diagnosing or monitoring a disease or condition (as mentioned herein) in a subject, which method comprises assessing the microcirculation in the limbus and optionally also the bulbar conjunctiva of said subject, said method comprising assessing the following parameter(s) in the subject's limbus and optionally also in their bulbar conjunctiva:
(i) functional capillary density (FCD); and/or
(ii) capillary flow velocity (CFV);
wherein parameters (i) and (ii) are assessed by microscopy.

By “monitoring” is meant assessing over a period of time, e.g. to monitor the progression of a subject's disease/condition over time, or in response to a treatment or treatment regimen, change of environment or other variable.

Viewed from a further aspect, the invention provides a method of determining the severity of a disease or condition (as mentioned herein) in a subject, said method comprising assessing the following parameter(s) in the subject's limbus and optionally also in their bulbar conjunctiva:
(i) functional capillary density (FCD); and/or
(ii) capillary flow velocity (CFV);
wherein parameters (i) and (ii) are assessed by microscopy.

In these aspects, preferably the subject is a subject with or suspected of having circulatory failure. “Circulatory failure” does not imply total failure, but the insufficient delivery of oxygen to the organs and cells of the body, sometimes in spite of full oxygen saturation of erythrocytes in the arteries. The circulatory failure identified or monitored may be systemic or localised. Examination of the limbus and BC is considered to provide a good indication of the systemic microcirculation.

Viewed from a further aspect, the invention provides a method of making a prognosis for a subject with a disease or condition (as mentioned herein), which method comprises assessing the microcirculation in the limbus and optionally also the bulbar conjunctiva of said subject, said method comprising assessing the following parameter(s) in the subject's limbus and optionally also in their bulbar conjunctiva:
(i) functional capillary density (FCD); and/or
(ii) capillary flow velocity (CFV);
wherein parameters (i) and (ii) are assessed by microscopy.

Viewed from a further aspect, the invention provides a method of making a prognosis for a subject with circulatory failure, the method comprising assessing the microcirculation in the limbus and optionally also the bulbar conjunctiva of said subject, said method comprising assessing the following parameter(s) in the subject's limbus and optionally also in their bulbar conjunctiva:
(i) functional capillary density (FCD); and/or
(ii) capillary flow velocity (CFV);
wherein parameters (i) and (ii) are assessed by microscopy.

Preferred embodiments of these methods are discussed earlier in relation to the first aspect of the present invention, namely a method of identifying or monitoring circulatory failure in a subject. Preferred embodiments and additional features of this first aspect, e.g. additional parameters (a) to (d), which are discussed above, apply mutatis mutandis to these later defined methods.

The methods of the invention all comprise assessing/analysing parameter(s) which result in values, i.e. measurement values for such parameters. These values are the results of the analysis. The assessing of parameters as recited elsewhere herein may comprise or consist of obtaining values for (or in respect of, or corresponding to) said parameters. A single value may be obtained for each parameter.

Preferably, the results of analysis are quantified by comparison to reference (or “control”) values. Such values are based upon values obtained from corresponding examinations of a reference (or “control”) subject or group of subjects. References herein to “reference subject” or “control subject” are intended to cover groups of such subjects as well as individual subjects. “Reference value” or “control value” therefore refers to value(s) obtained from groups of reference/control subject(s) or from individual subjects.

In any of the embodiments herein, a value “for” a parameter can be, alternatively or in addition, a value “corresponding to” or “in respect of” said parameter.

The subject of the method of the invention, i.e. the subject whose data is ultimately compared with said reference/control subject's data, is termed the “patient subject”.

Thus, preferably any method of the invention comprises the step of comparing the results of the analysis of the assessed parameters with reference/control values for the same parameters.

Preferably, the method of the invention comprises determining the deviation between the value(s) obtained for (or measurements of) the assessed parameters in a subject and a corresponding healthy reference value, value of a healthy control, or value of the subject from an earlier point in time, wherein (typically) a statistically significant deviation indicates circulatory failure in said subject.

Reference or control values are obtained from the corresponding assessment of a reference or control subject. “Corresponding assessment” means that the reference/control values are or have been obtained from the same conjunctival area in the reference/control subject(s), using the same methodology as for the patient subject.

Preferably the reference subject is a “healthy reference subject”, i.e. the values are “healthy reference values”. In this scenario, positive findings (e.g. healthy circulation) are indicated by a lack of significant deviation, preferably a lack of deviation, in the microcirculatory parameter values from the patient subject and the reference subject. A deviation, in particular a significant deviation, indicates negative findings (e.g. circulatory failure). Many statistical methods are known to establish deviations which are considered significant. In general, a deviation which is at least 10%, 20%, 30% or 40% from a healthy reference value is significant and typically is indicative of CF. The greater the degree of deviation, the more negative the findings. Generally, the more parameters that deviate from healthy reference values, the more negative the findings are.

Alternatively, the reference subject may be a subject (group of subjects) with a known disease state or condition (“pathological reference subject/values”). The disease/condition is preferably selected from those mentioned elsewhere herein. In this scenario, negative findings (e.g. presence of disease) are indicated by a lack of significant deviation, preferably a lack of deviation, in the microcirculatory parameter values from the patient subject and the reference subject. A significant deviation indicates positive findings (e.g. absence of disease). The greater the degree of deviation, the more positive the findings. The smaller the degree of deviation, the more negative the findings. Generally, the fewer parameters that deviate from pathological reference values, the more negative the findings are.

Preferably test results can be compared against a database of reference values or against threshold values obtained from a database, i.e. from previously obtained values. Alternatively, any method of the invention may comprise the active step of assessing parameters (a) to (f) on reference/control subjects in order to obtain said reference/control values.

Reference/control values may also have been obtained from the same subject at an earlier point in time, which is of particular use in monitoring the progression of a disease or condition in said subject over time, or a response to treatment, change of environment or other variable. This approach also permits monitoring the efficacy of a treatment regime. In this scenario, for example where a therapeutic intervention is being assessed, a deviation from historic/reference values in that subject may be a positive outcome.

The invention thus extends to obtaining information that may be useful when monitoring the effect of treatment on the subject. Typically the assessment will require an assessment before and after (optionally also during) the intervention and comparison of the results obtained with one another and/or with reference values. The effectiveness of the intervention will generally be positively correlated with its ability to result in, or tend towards, a normal microcirculation. By repeated assessments according to the invention, the effect of the specific therapy on the patient can be evaluated.

Thus in a further aspect, the present invention provides a method of monitoring the efficacy of treatment in a subject, the method comprising assessing the microcirculation in the limbus and optionally also the bulbar conjunctiva of said subject, said method comprising assessing the following parameter(s) in the subject's limbus and optionally also in their bulbar conjunctiva:
(i) functional capillary density (FCD); and/or
(ii) capillary flow velocity (CFV);
wherein parameters (i) and (ii) are assessed by microscopy; and
comparing the results of the assessment to the results of the corresponding assessment obtained from the same subject at an earlier point in time.

The earlier point in time may be prior to the commencement of treatment, or an earlier point in time during the treatment.

The treatment may be administration of a therapeutic agent, performance of a therapeutic regimen, or any other therapeutic step.

Today the scientific basis for selection of therapy is based on understanding of the disease mechanism (pathogenesis) as well as results from evidence based studies. To prove statistically that Therapy A is more effective than Therapy B in a double blind, randomized and placebo controlled study in a cohort of patients, only a minority of patients treated with Therapy A may have a better outcome as compared to patients treated with Therapy B. But Therapy A may be of no value to a majority of the patients in the cohort, or even be harmful to some patients. The present invention may be used to identify which patients show improved oxygen delivery to the cells of the body after start of the therapy (by trend analysis of repeated measurements before and after start of the therapy). Likewise, it will be possible to identify patients where Therapy A is harmful and where Therapy B is beneficial. The present invention provides for the design of an optimal individualised treatment. As a result of this assessment of effectiveness of treatment, i.e. progress made by the patient, the clinician may then decide to continue, cease or alter the intervention.

Therefore the results of these microvascular examinations can be used to improve selection of appropriate treatment and/or to guide or monitor therapy.

Using trend analyses from repeated assessments taken before and after start of a specific treatment for identified circulatory failure the responders to the treatment can be identified and in non-responders the treatment can be stopped.

All methods of the invention described herein, comprise assessment steps wherein a microscope and optionally a spectrometer are used to analyse the microcirculation in the limbus and optionally the BC of a subject. As a result of these assessments, optionally utilising a comparison with reference values, information regarding circulatory failure and likely clinical outcome is obtained. Such information may give or may contribute to a diagnosis or prognosis for the subject. As a consequence, a therapeutic step may be taken, in particular to cease, continue or alter a therapeutic intervention or regimen. Obtaining information, making a diagnosis or prognosis and a consequential therapeutic step are steps which make up further embodiments of the methods of the invention.

Thus, the methods, preferably comprise a step, subsequent to said assessment and any comparison to reference values, of deciding to start a therapeutic intervention or regimen, or to cease, continue or alter a therapeutic intervention or regimen which the subject is receiving.

The methods preferably comprise a step of making a diagnosis. The diagnosis may be a diagnosis of circulatory failure. The diagnosis may be based wholly or partially on the findings of the method of the invention. For example, the diagnosis may be based on the assessment steps of the method of the invention, for example value(s) of the parameter(s) obtained from said assessment or the comparison of said value(s) to reference value(s). The diagnosis may be based on an indication (e.g. an indication of circulatory failure) derived from the aforementioned assessment, value(s) and/or comparison. Alternatively worded, the diagnosis of a disease may be made when said disease (e.g. circulatory failure) is indicated from the aforementioned assessment, value(s) and/or comparison.

The methods preferably comprise a step, subsequent to said assessment and any comparison to reference values and/or making of a diagnosis, of starting a therapeutic intervention or regimen, or ceasing, continuing or altering a therapeutic intervention or regimen which the subject is receiving.

As discussed above, information related to oxygen saturation in the limbus and optionally also the BC can give valuable insight into the microcirculation and general circulatory health and related diseases in a subject. In a further aspect, the present invention provides a method of identifying or monitoring circulatory failure in a subject, which method comprises assessing the microcirculation in the limbus and optionally also the bulbar conjunctiva of the subject, said method comprising assessing the following parameter(s) in the subject's limbus and optionally also in their bulbar conjunctiva:
(c) oxygen saturation of microvascular erythrocytes (SmvO₂); and optionally also
(d) heterogeneity of SmvO₂ in the limbus.

Parameters (c) and (d) may be assessed in conjunction with one or both (preferably both of) parameters (i), (ii) and optionally also parameters (a) and (b).

Other methods (e.g. of prognosis) and preferred features discussed above in relation to methods of assessing parameters (i) and (ii) apply mutatis mutandis to this aspect of the invention.

The invention also extends to apparatus for carrying out the methods discussed herein. Thus, viewed from a further aspect, there is provided apparatus for assessing microcirculation in the limbus of a subject comprising a microscope, optionally a spectrometer, and a computer, whereby the computer is arranged (or configured) to receive image(s) of the microcirculation obtained using the microscope and optionally data relating to SmvO₂ from the spectrometer and, optionally, to process the image(s) and data to identify and/or determine characteristics/parameters associated with pathology, wherein the image(s) and data relate to the following parameters:
(i) functional capillary density (FCD); and/or
(ii) capillary flow velocity; and optionally one or more of:
(a) heterogeneity of the FCD;
(b) heterogeneity of CFV;
(c) oxygen saturation of microvascular erythrocytes (SmvO₂); and
(d) heterogeneity of SmvO₂.

Preferably the apparatus is also for assessing microcirculation in another conjunctival region of the subject, preferably the bulbar conjunctiva.

Software for analysis of the collected frames/films and the spectrometer curves can be installed on the same computer, but analysis may also be performed on a separate computer after the collected files have been transferred to this other computer. The first computer (receiving computer) can have installed software for real time analysis, both for DRS and for CAVM files. Preferably, offline analysis is performed on another computer (processing computer), just as in a professional biochemical lab that receives blood samples from a general practitioner (GP).

The microscope is preferably used to obtain the image(s) from the limbus and optionally also other parts of a subject's conjunctiva, preferably the bulbar conjunctiva, and is then removed therefrom prior to the processing steps. Likewise, a probe attached to the spectrometer is optionally used on the limbus and optionally also other parts of a subject's conjunctiva, preferably the bulbar conjunctiva, and then removed prior to the processing steps.

Indeed, a further aspect of the invention relates to the apparatus for processing previously acquired data. Thus, viewed from a still further aspect there is provided apparatus for assessing microcirculation in the limbus of a subject comprising a computer arranged (or configured) to receive image(s) of the microcirculation of a subject's limbus obtained using a microscope and optionally data relating to SmvO₂ from a spectrometer and to process the image(s) and data to identify and/or determine characteristics/parameters associated with pathology, wherein the image(s) and data relate to the following parameters:
(i) functional capillary density (FCD); and/or
(ii) capillary flow velocity; and optionally one or more of:
(a) heterogeneity of the FCD;
(b) heterogeneity of CFV;
(c) oxygen saturation of microvascular erythrocytes (SmvO₂); and
(d) heterogeneity of SmvO₂.

Preferably the apparatus is also for assessing microcirculation in another conjunctival region of the subject, preferably the bulbar conjunctiva, and comprises a computer arranged (or configured) to receive image(s) of the microcirculation of the subject's other conjunctival region, preferably the bulbar conjunctiva, obtained using a microscope and optionally also data relating to SmvO₂ from a spectrometer, and to process the image(s) and data to identify and/or determine characteristics/parameters associated with pathology.

The apparatus preferably further comprises a means for outputting values corresponding to the characteristics/parameters and/or a value based on them in combination, such as a weighted sum or average. Such a value may be regarded as a microvascular pathology score. An algorithm can be used to process the raw data such that each of the parameters defined herein is given a weight, weights may be optimised for different patient cohorts, e.g. for premature or for full term neonates. The output value of the algorithm typically corresponds to a weighted sum or average.

The invention also extends to software, whether in tangible form on a data carrier, or downloadable via a network, comprising instructions to cause a computer to carry out the processing and/or output steps mentioned above.

The invention further extends to the use of such an apparatus and/or such software to assess the subject's microcirculation in the limbus, preferably also the microcirculation in the bulbar conjunctiva of said subject, and based on those assessments/measurements to provide clinically, e.g. diagnostically, relevant information about a subject, to diagnose a subject, to determine the severity of a disease or condition in a subject, to make a prognosis for a subject with a disease or condition, and/or to monitor the efficacy of treatment in a subject.

In a further aspect the present invention provides a computer implemented method of performing any of the methods (or method steps) described elsewhere herein. Hence, any method (or method step) described elsewhere herein may be computer implemented (or implemented by a computer, or implemented using a computer, or performed by computer, or performed using a computer).

The invention will now be described in more detail by reference to the following non-limiting Figures and Examples, in which:

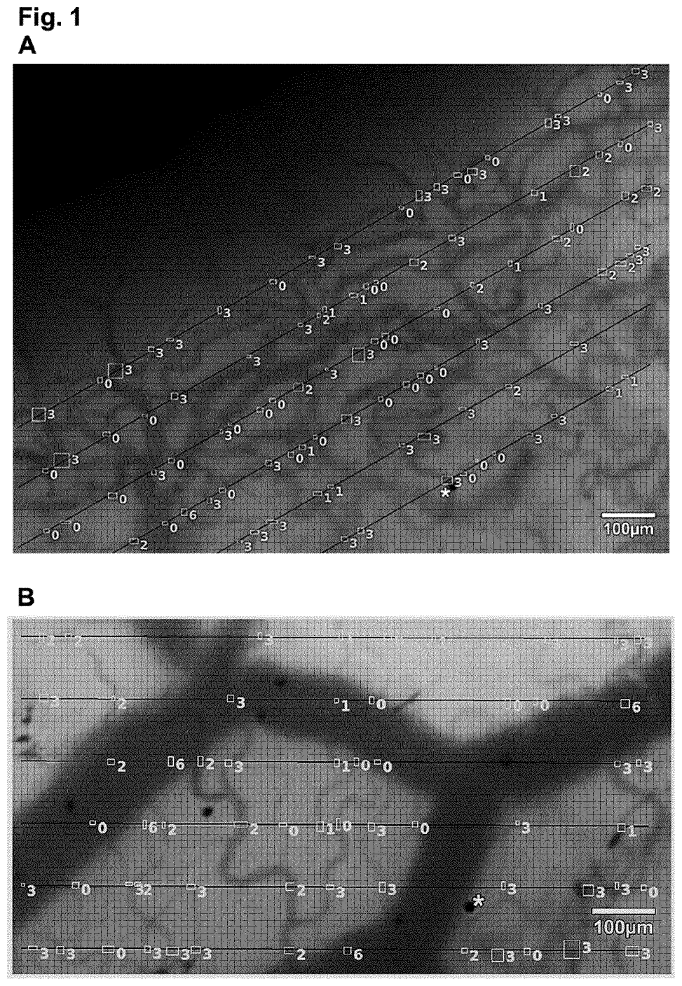

FIG. 1 shows microscopy images. 1A: Frame from limbus. Avascular cornea seen in the upper left corner. 1B: Frame from bulbar conjunctiva. In 1A and 1B, boxes identify capillaries crossing six parallel lines. The number beside each box represents capillary flow velocity category. Vessels larger than 20 microns are not included in the analyses. Black dots (*) seen in both frames are melanin aggregates.

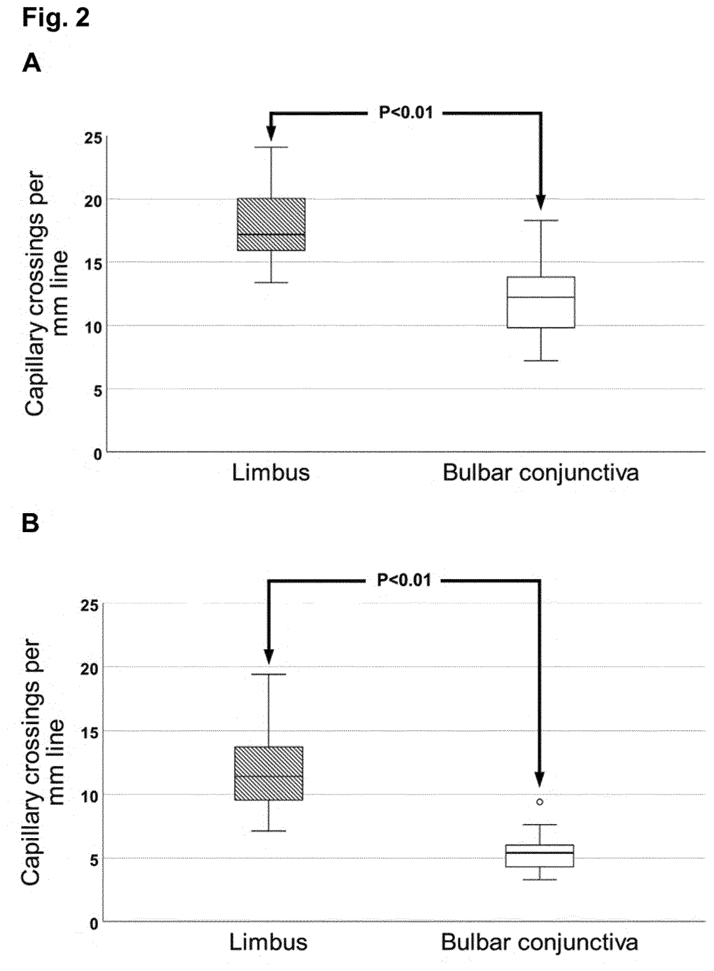

FIG. 2 shows functional capillary density at limbus and bulbar conjunctiva in Oslo University Hospital (2A) and Cleveland Clinic (2B).

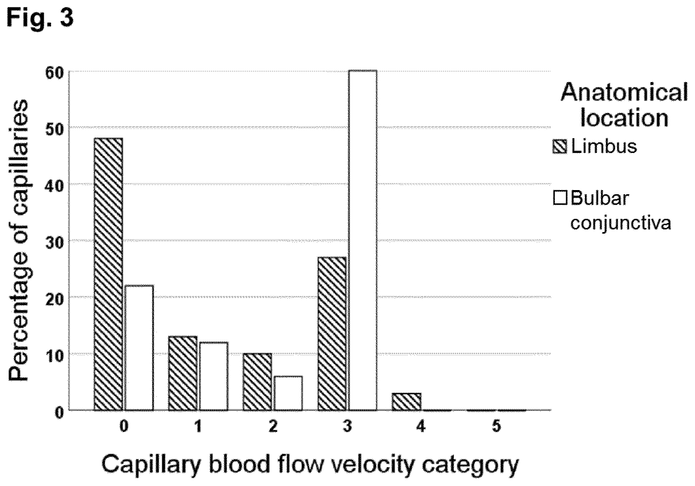

FIG. 3 shows capillary flow velocity distribution at the limbus and bulbar conjunctiva (Cleveland Clinic recordings).

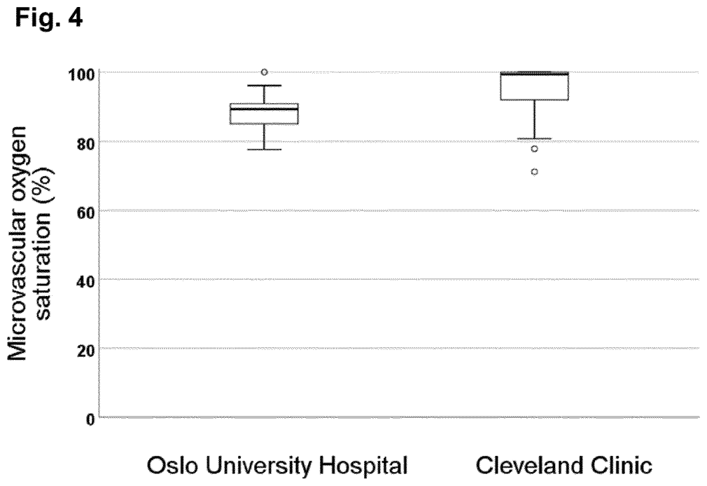

FIG. 4 shows conjunctival microvascular oxygen saturation data from Oslo University Hospital and Cleveland Clinic.

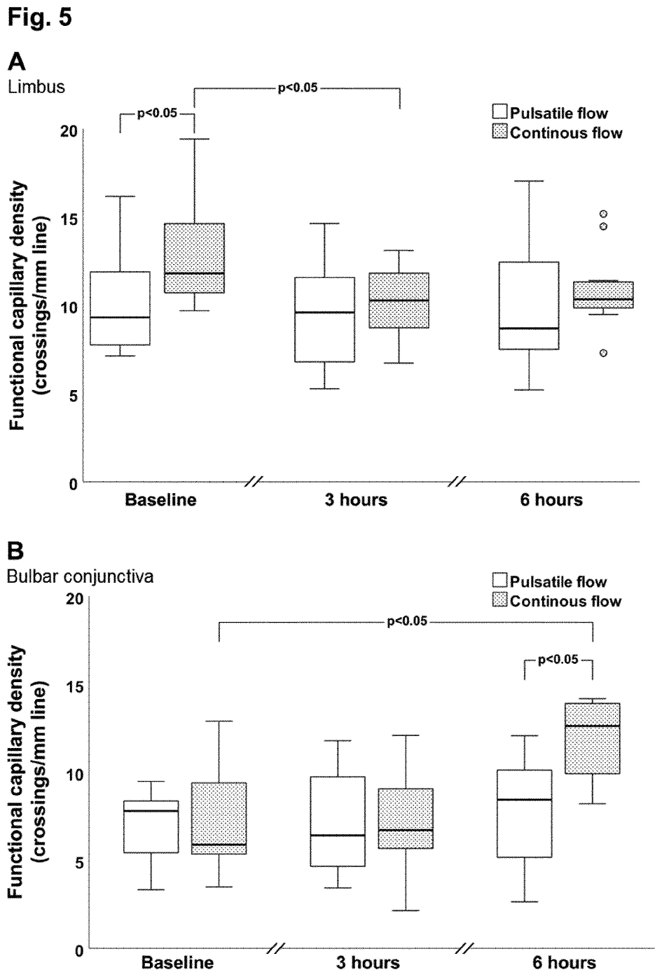

FIG. 5 shows functional capillary density (FCD) at baseline, 3 hours and 6 hours in piglets having slowly deteriorating circulatory health (piglets on a pulsatile flow pump) and piglets having more rapidly deteriorating circulatory health (piglets on a continuous flow pump), in limbus (FIG. 5A) and bulbus (FIG. 5B).

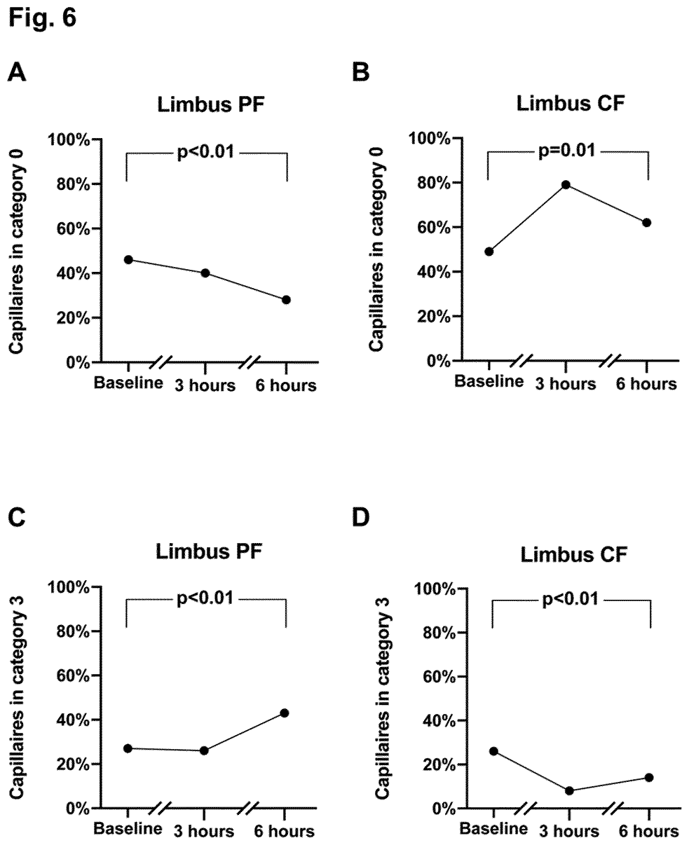

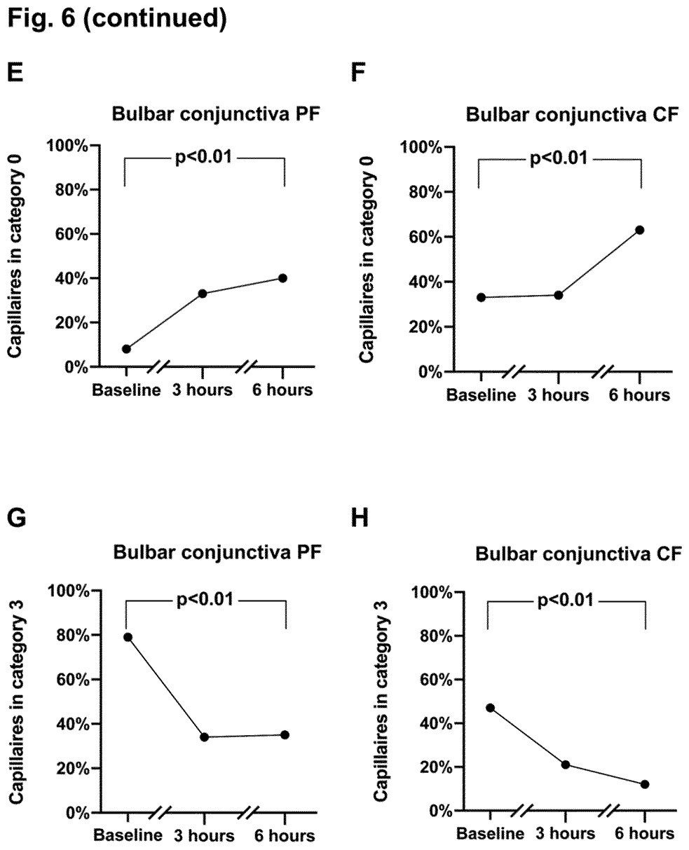

FIG. 6 shows capillary flow velocity patterns in limbus (panels A to D) and bulbus (panels E to H). Dots represent percentage of all recordings for each pump at baseline, three and six hours, and are connected with a line to visualize trends. PF=pulsatile flow, CF=continuous flow.

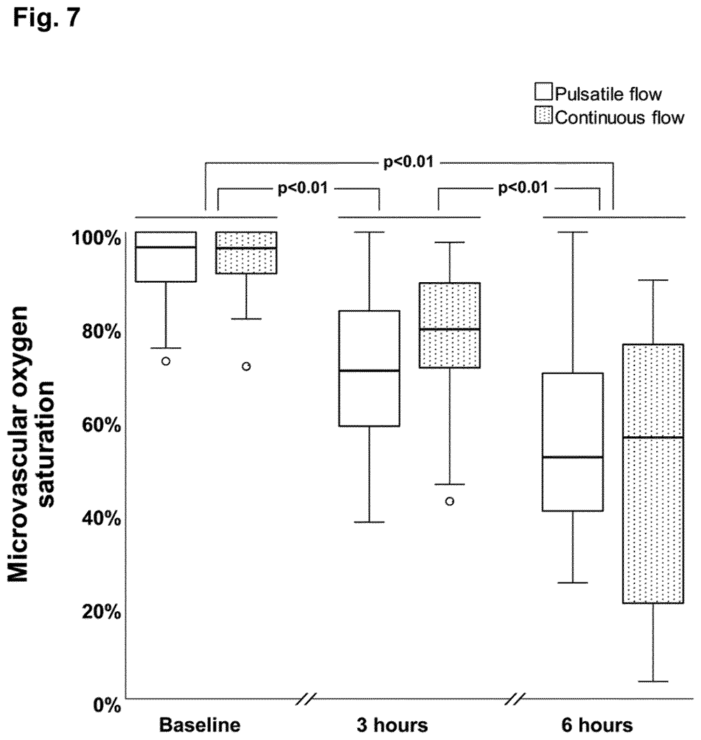

FIG. 7 shows measurements of microvascular oxygen saturation (SmvO₂) in piglet models of slowly deteriorating circulatory health (piglets on a pulsatile flow pump) and more rapidly deteriorating circulatory health (piglets on a continuous flow pump).

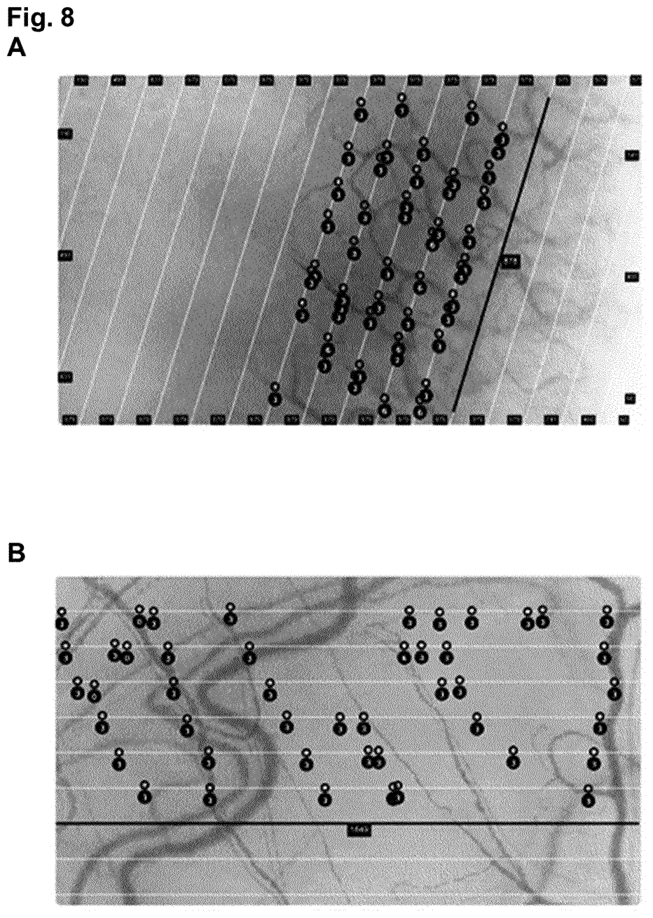

FIG. 8 shows frames from limbus (A) and bulbar conjunctiva (B) of human subjects during analysis in Example 3.

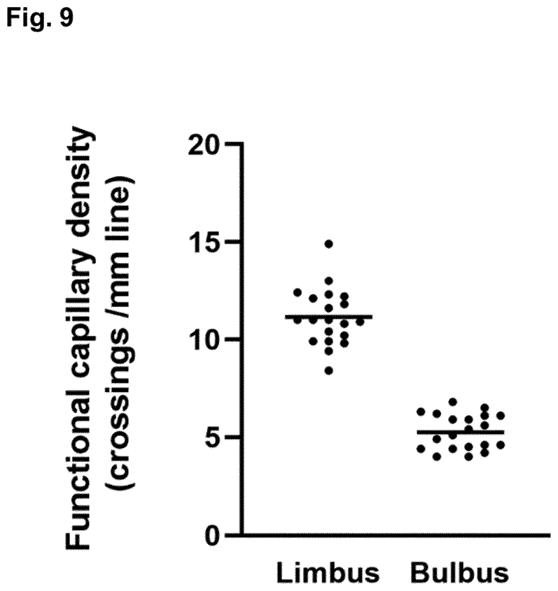

FIG. 9 shows ocular surface functional capillary density measured in the limbus and bulbar conjunctiva of healthy human subjects. Each dot represents the mean of valid recordings from one volunteer. Horizontal lines represent the mean of each data set.

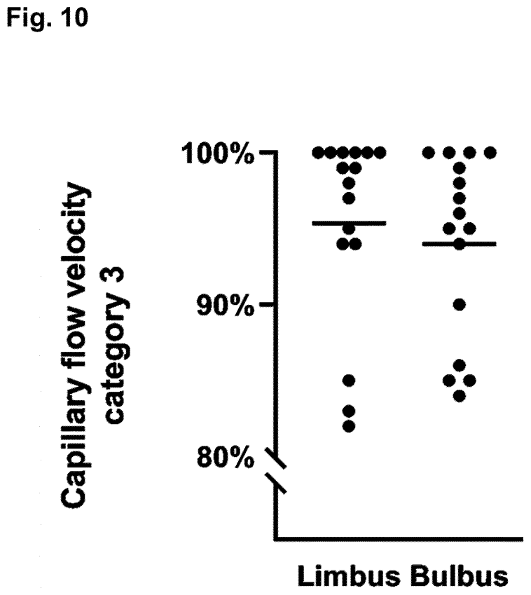

FIG. 10 shows ocular surface capillary flow velocity measured in the limbus and bulbar conjunctiva of healthy human subjects. Each dot represents the mean of valid recordings from one volunteer. Horizontal lines represent the mean of each data set.

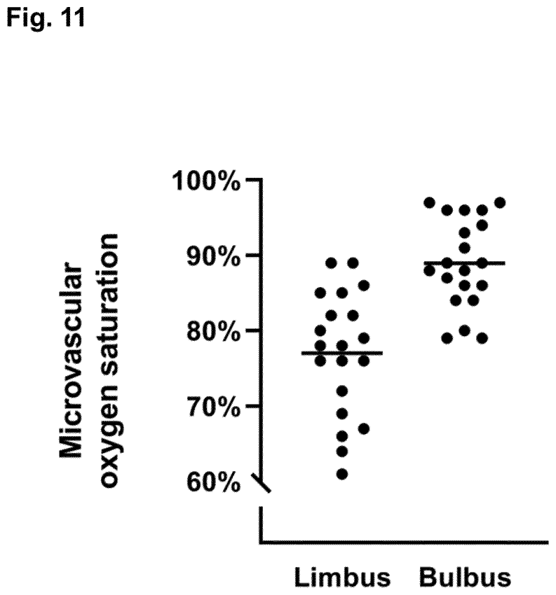

FIG. 11 shows ocular surface microvascular oxygen saturation measured at limbus and in bulbar conjunctiva of healthy human subjects. Each dot represents the mean value within one individual's recordings. Horizontal lines represent the mean of each data set.

### EXAMPLES

Abbreviations

| | |
|---|---|
| CF | continuous flow |
| PF | pulsatile flow |
| CPB | cardio-pulmonary bypass |
| ECMO | extra-corporeal membrane oxygenation |
| PP | perfusion pressure |
| VR | vascular resistance |
| CFV | capillary flow velocity |
| FCD | functional capillary density |
| ODIN | Oxygen delivery index |
| SmvO₂ | microvascular oxygen saturation |
| CC | Cleveland Clinic |
| OUH | Oslo University Hospital |

### Example 1—Piglet Study Assessing the Limbus and Bulbar Conjunctiva

### Material and Methods

### Study Population

Experiments were performed in two laboratories with common research interests: Oslo University Hospital (OUH), Norway and Cleveland Clinic (CC), USA. The conjunctival microcirculation of five Norwegian landrace piglets of both sexes (30±3 kg) (OUH) and eight female Yorkshire piglets (44±4 kg) (CC) was recorded. The study at OUH was approved by the Norwegian Food Safety Authority (FOTS, id: 6689). At CC, the study protocols were approved by Institutional Animal Care and Use Committee (study number 2016-1659). The animals received humane care in compliance with the Guide for the Care and Use of Laboratory Animals and institutional guidelines.

### Equipment for Microvascular Examinations

Both study sites used a mobile microvascular laboratory (mLab, version α1, ODI Medical AS, Oslo, Norway), containing a digital video microscope, a spectroscope and a computer with custom made software.

### Digital Microscopy

A digital hand-held video-microscope (Optilia, D1, Instruments AB, Sollentuna, Sweden) with a 300× magnification lens, image resolution of 1920×1080 pixels, field of view of 1.13 mm×0.7 mm and frame rate of seven frames per second was used to obtain conjunctival recordings of 20 seconds duration.

One operator (AKK) conducted analyses off-line (EyeSoft version 1.0, ODI). Adjustments of light intensity, contrast and tint optimized the image quality. In limbal recordings, a line touching the edge of the cornea was created by the operator. An additional five parallel lines separated by 100 μm were created by the software at increasing distances from the cornea (FIG. 1A). In films from the bulbar conjunctiva, six horizontal lines separated by 100 μm were drawn (FIG. 1B).

Capillaries (blood vessels <20 μm) containing erythrocytes crossing the lines were identified and marked manually. Empty capillaries are not seen by the microscope. FCD was expressed as capillary crossings/mm line (c/mm).

In the CC series, CFV at each capillary crossing was determined according to a six-category velocity scale (Table 1). If the velocity fluctuated during a film sequence, the average velocity throughout the film was used. Data were expressed as the fraction of capillaries in each blood flow category.

### Spectroscopy

The Diffuse Reflectance Spectroscopy (DRS) system used in this study has a measuring volume in the range of 0.1 mm³ (Sundheim et al., 2017). A halogen light source (AvaLight-HAL, Avantes, Apeldoorn, The Netherlands) with spectral range 450-800 nm, and a measuring probe (FCR-7uv400-2.3-bx, Avantes) with six emitting and one receiving 400 μm fibers were connected to a spectrometer (AvaSpec-2048-2, Avantes). Calibration was performed before and after each set of measurements by holding the probe against a white polytetrafluorethylene tile (WS-2, Avantes).

The DRS spectra analyses were carried out with the help of a simplified tissue model that calculates light propagation in the tissue, using the approach from Farrell (Farrell et al., 1992) and Jacques (Jacques, 2013). Starting with a set of initial propagation parameters for the tissue, the model implemented in Python/MATLAB, produced a spectrum that is compared to the acquired DRS to calculate the optimal propagation parameters that reproduce the best fit to the data. Finally, the oxyhemoglobin and deoxyhemoglobin concentrations were calculated.

### Pulse Oximetry

The arterial oxygen saturation was monitored by a pulse oximeter attached to the tail (OUH: Capnostream 20p, Oridion Medical, Israel) or the ear (CC: Aisys CS2, GE healthcare, Illinoi U.S.).

### Laboratory Procedure

In the present study, the limbus is referred to as the surface of the eye where the transparent corneal and the opaque vascularized conjunctival tissue fuse; the limbus is the region less than 1 mm from the cornea (i.e. from the corneal/limbal boundary). The bulbar conjunctiva was defined as the conjunctival tissue located at least 2 mm away from the cornea (i.e. from the corneal/limbal boundary).

At both study sites, conjunctival microvascular baseline data were collected from the left eye following general anesthesia and prior to other planned experiments.

At OUH, the piglets acclimatized in a cubicle with free access to water and food three to six days prior to the study. The piglets were sedated by injecting narketan/ketamine (11/33 mg/kg) intramuscularly in the neck. Fentanyl (30-100 μg/kg/h) and propofol (12-20 mg/kg/h) injections induced general anesthesia. The piglets were fixed in a supine position and orotracheally intubated. When stable in general anesthesia, a tracheostomy was performed, and an endotracheal tube inserted. Venous access for infusions and blood samples were secured by a catheter in the jugular vein. A catheter in the common carotid artery was used for monitoring blood pressure and sampling for blood gas analyses. A surgically inserted catheter in the urinary bladder measured urinary output and body temperature. Following surgery, the animals were placed on the right side before data acquisition. Finishing all experiments, the animals were euthanized by IV injection of potassium chloride.

At CC, the animals were quarantined and monitored for at least three days prior to the study. Each piglet fasted for 12 hours prior to surgery. As pre-medication, xylazine (2 mg/kg, IM) and ketamine (20 mg/kg, IM) were administered. Propofol (1 mg/kg, IV) or buprenorphine (0.05 mg/kg, IV) induced general anesthesia. The piglets had an endotracheal tube installed, and anesthesia was maintained by isoflurane gas (1.0-3.0%). The temperature was monitored by a rectal thermometer (Aisys CS2 GE healthcare, Illinoi, U.S.). The animal was placed in a supine position, and then given lidocaine (up to 2 mg/kg/h IV) to prevent ventricular arrhythmia. At the end of the studies, the animal was heparinized (500 U/mg) and sacrificed by administration of potassium chloride (2.5-3.5 mEq/kg, IV) under deep anesthesia with isoflurane (5%).

### Conjunctival Microvascular Measurements

The same protocol was used for data acquisition at the two study sites. Microscopy films were recorded from two locations: The limbus (<1 mm from the cornea, i.e. cornea seen in part of the frames) and the bulbar conjunctiva (>2 mm away from the cornea). Isotone saline was applied to the eye prior to recordings. At least four film sequences were recorded from both locations. DRS spectra were collected from the conjunctiva.

### Statistics

Functional capillary density and SmvO₂ results are presented as mean±standard deviation and displayed as box-whisker plots. Comparing FCD from the limbus and the bulbar conjunctiva, an independent samples t-test, with a significance level of 5% was used. CFV data are presented as median with interquartile range and displayed in a histogram. Mann Whitney U test was applied to compare CFV data from limbus and bulbus. The statistical analysis was performed by SPSS version 25 (SPSS Inc, Chicago, Illinois, USA).

### Results

It was possible to obtain microscopy films and DRS spectra from all piglets both at OUH and CC. No adverse events were recorded during data collection.

### Functional Capillary Density (FCD)

119 films were recorded, of which 47 were of inferior image quality and excluded from analyses. 3,422 capillaries were analyzed (Table 2). At both study sites, the FCD was higher at limbus as compared with the bulbar conjunctiva (OUH: 18.1±2.9 versus 12.2±2.9 c/mm, p <0.01, CC: 11.3±3.0 versus 7.1±2.8 c/mm, p<0.01) (FIG. 2).

### Capillary Flow Velocity (CFV)

The most prominent flow categories were 0 (no flow) in limbus and 3 (continuous flow) in bulbar conjunctiva (FIG. 3). Median categorical CFV was higher in bulbar recordings as compared with limbal recordings (CC: 3 (1-3) versus 1 (0-3), p<0.01).

### Microvascular Oxygen Saturation (SmvO₂)

All piglets at both study sites had pulse-oximetry assessed arterial oxygen saturation ≥95%. Twenty-nine DRS spectra from Oslo and 91 from Cleveland were analyzed (FIG. 4). Mean microvascular oxygen saturation was high at both study sites (OUH: 88±5.9%, CC: 94±7.5%).

TABLE 1
Categories of capillary flow velocities

| Blood flow | |
|---|---|
| category | Description of blood flow |
| 0 | No flow: Visible erythrocytes, no movement |
| 1 | Sluggish flow: Intermittent slow flow |
| 2 | Slow flow: Continuous slow flow or Intermittent “no flow” |
| | and rapid flow |
| 3 | Continuous flow |
| 4 | Rapid flow: Continuous rapid flow or Intermittent slow and |
| | brisk flow |
| 5 | Brisk flow: Continuous brisk flow |
| 6 | Uncertain: Velocity impossible to determinate |

TABLE 2
Number of microscopy films and analyzed capillaries. Capillary blood
flow velocity is only scored in data from Cleveland Clinic and not
in the Oslo University Hospital series due to quality of the films.

| | | OUH | CC |
|---|---|---|---|
| Total number of recorded films | | 49 | 70 |
| Number of analyzed films | Limbus | 19 | 24 |
| | Bulbar conjunctiva | 17 | 12 |
| Number of analyzed | Limbus | 1533 | 526 |
| capillaries | Bulbar conjunctiva | 1135 | 228 |

### Discussion

The present study indicates that non-invasive digital video microscopy and diffuse reflectance spectroscopy can be used to obtain data from conjunctival microcirculation. In a piglet model, two independent sites found that limbal conjunctival microcirculation had a larger capacity for oxygen delivery as compared with bulbar conjunctiva measured by the method of the present invention.

The conjunctiva provides a window of examination in patients with systemic or local eye circulatory failure (e.g. glaucoma, diabetes and ischemia). In ophthalmology, one could use the method of the present invention to quantify ocular surface injuries, in guiding treatment options and predict healing potential. Given that the retina and bulbar conjunctiva share the same embryological ectodermal origin and vascular supply, the microcirculation of the conjunctiva may also mirror the microcirculation of the central nervous system in patients with both systemic circulatory failure and brain pathology (e.g. ischemic stroke or high intracranial pressure).

In this study there was higher FCD in limbal recordings as compared with bulbar recordings. The bulbar FCD in the OUH piglets of 12.2±2.9 c/mm was comparable to reported findings in the groin skin of piglets of the same breed 10.4 and 13.2 c/mm. The FCD results reported in this study are also within the same range as results from skin of the hand (12.4±1.6 c/mm) and chest (10.7±1.6 c/mm) of newborns.

Differences between the two conjunctival regions were found at both study sites, supporting that the method is able to detect a higher metabolic rate in limbus as compared to bulbus.

### REFERENCES

Farrell, T. J., Patterson, M. S., Wilson, B., 1992. A diffusion theory model of spatially resolved, steady-state diffuse reflectance for the noninvasive determination of tissue optical properties in vivo. Medical physics 19, 879-888.
Jacques, S. L., 2013. Optical properties of biological tissues: a review. Physics in medicine and biology 58, R37-61.
Sundheim, L. K., Sporastøyl, A. H., Wester, T., Salerud, G., Kvernebo, K., 2017. Acute skin trauma induces hyperemia, but superficial papillary nutritive perfusion remains unchanged. Microcirculation (New York, N.Y.: 1994) 24.

### Example 2—Study Monitoring the Conjunctival Microcirculation of Piglets Having Slowly Deteriorating Circulatory Health (Piglets on PF Pumps) or More Rapidly Deteriorating Circulatory Health (Piglets on CF Pumps) Using the Analytical Method of the Present Invention

### Material and Methods

In sleeping piglets, we examined the effect of 6 hours of continuous flow (CF) and pulsatile flow (PF) cardiopulmonary bypass (CPB) on the microcirculatory function in limbal and bulbar conjunctiva. Pulsatile flow is a better approximation of normal circulation than continuous flow but animals attached to this type of pump are still not normal/healthy and over time will tend to exhibit symptoms of impaired circulation (for example the medication given to the pigs may induce narcosis and affect microvascular perfusion).

### Study Population

Eight healthy female Yorkshire piglets (44±4 kg); five of which were supported on PF pump (VentriFlo True Pulse Pump®, Design Mentor Inc., Pelham, NH, USA) and three on a CF pump (Roatflow®, Maquet Holding B.V. & Co. KG, Rastatt, Germany) were examined. The study protocol (number 2016-1659) was approved by Institutional Animal Care and Use Committee at Cleveland Clinic. The piglets received humane care in compliance with the Guide for Care and Use of Laboratory Animals and institutional guidelines.

### Methods

Computer assisted video microscopy (CAVM) and diffuse reflectance spectroscopy (DRS) were used to record microvascular data in small tissue volumes (˜0.1 mm³) from two locations in the conjunctiva: limbus and bulbar conjunctiva.

### Computer Assisted Video Microscopy

A 300× magnification lens attached to a digital microscope (Optilia, D1, Instruments AB, Sollentuna, Sweden) with autofocus, 1.13 mm×0.7 mm field of view, framerate of seven frames per second and image resolution 1920×1080 pixels, recorded films of 20 seconds duration. Film analyses were performed off-line using a custom-made software (EyeSoft version 1.0, ODI Medical, Oslo, Norway. Capillaries, defined as visible blood vessels <20 μm, crossing six parallel lines separated by 100 μm were identified. Functional Capillary Density (FCD) was expressed as capillary crossings/mm line. Capillary Flow Velocity (CFV) at each crossing was determined according to a six-category velocity scale ranging from category 0 (no flow) to category 5 (brisk flow) (the categories 0 to 5 are the same categories 0 to 5 defined in Table 1).

### Diffuse Reflectance Spectroscopy (DRS)

A spectrometer (AvaSpec-2048-2, Apeldoorn, The Netherlands) with a tungsten halogen light source (AvaLight-HAL, Apeldoorn, The Netherlands) with spectral range of 450 to 800 nm, was used for examination of microvascular oxygen saturation (SmvO₂). The fiber optic probe was calibrated against a white polytetrafluorethylene tile (WS-2, Avantes, The Netherlands) prior to each set of recordings. DRS spectra were analyzed by a custom made algorithm (EyeSoft version 1.0, ODI Medical) to calculate SmvO₂.

### Lab Procedure

The piglets were quarantined and monitored at the laboratory facility for at least 3 days and fasted 12 hours before surgery at Cleveland Clinic, Ohio, USA. Vital signs (respiratory rate, appetite, general condition) were monitored. Xylazine (2 mg/kg, intramuscularly (IM)) and ketamine (20 mg/kg, IM) were given as pre-medication. Anesthesia was induced by propofol (1 mg/kg, intravenously (IV)) or buprenorphine (0.05 mg/kg, IV) and maintained by volatile administration (isoflurane 1.0-3.0%) throughout the experiment.

Fixated in a supine position with electrocardiogram leads attached to the extremities, the neck, groin, and chest were prepared for open chest surgery. Lidocaine (<2 mg/kg/h IV) was given to prevent ventricular arrhythmia. Arterial monitoring lines were inserted in the carotid artery and venous pressure line in the jugular vein. The pericardium was opened following a median sternotomy. Cardiac output was measured by a pulmonary artery flow probe. Cannulas were inserted into the ascending aorta (22 Fr EOPA® [elongated one-piece] Arterial Cannula, Medtronic Perfusion Systems, Minneapolis, MN, or 21 Fr Arterial Cannula, Edwards Lifesciences, Irvine, CA) and the inferior vena cava (34-46 Fr or 29-37 Fr MC2 Two-Stage Cannula, Medtronic Perfusion Systems, Brooklyn Park, MN) accessed through the right atrial appendage. A vent tube inserted in the left atrium or ventricle prevented ejection of blood. The pulmonary artery flow was kept at 0 L/min.

When activated clotting time exceeded 450 s following heparin administration (500 IU/kg, IV), CPB with either PF (VentriFlo True Pulse Pump®) or CF (Roatflow®) was engaged at a flow rate of 50 ml/kg/min and maintained for 6 hrs. Quadrox (Maquet Holding B.V. & Co. KG, Rastatt, Germany), Affinity Fusion (Medtronic, Minnesota, USA) or Capiox (Terumo Cardiovascular Systems, Tokyo, Japan) oxygenators were used in the circuit. Vasoactive agents were not administered during 6-hr CPB. Arterial blood gas sample was drawn hourly. After 6 hours on CPB under deep anesthesia (isoflurane 5%), the piglets were subjected to an additional dose of heparin (500 U/mg) and sacrificed by a lethal dose of potassium chloride (2.5-3.5 mEq/kg, iv).

### Recording Procedure

The eyelids of the left eye were retracted by sutures during measurements and were closed between recordings. Saline was applied to maintain a moist conjunctival surface between measurements. At least four film sequences of 20 seconds were recorded from limbus and the bulbar conjunctiva at baseline, three and six hours. DRS spectra were collected from the conjunctiva. The equipment was moved slightly between each recording in order to capture different measuring volumes.

During data collection, each recorded file was labeled by a code. The analyzer was blinded to type of heart pump and time of recording. Prior to statistical analysis, the identity of the piglet, pump type and time was revealed to the analyzer.

An overview of numbers and location of analyzed capillaries and optical spectra is given in Table 3.

TABLE 3
Overview of data content.

| Anatomical | | Baseline | | 3 hours | | 6 hours | |
|---|---|---|---|---|---|---|---|
| location | Parameter | PF | CF | PF | CF | PF | CF |
| Limbus | FCD | 224 | 302 | 217 | 318 | 252 | 279 |
| | (number of | | | | | | |
| | assessed | | | | | | |
| | capillaries) | | | | | | |
| | CFV | 103 | 182 | 166 | 212 | 176 | 229 |
| | (number of | | | | | | |
| | assessed | | | | | | |
| | capillaries) | | | | | | |
| Bulbus | FCD | 101 | 107 | 107 | 135 | 531 | 52 |
| | (number of | | | | | | |
| | assessed | | | | | | |
| | capillaries) | | | | | | |
| | CFV | 75 | 101 | 96 | 130 | 370 | 49 |
| | (number of | | | | | | |
| | assessed | | | | | | |
| | capillaries) | | | | | | |
| Conjunctiva | SmvO₂ | 55 | 35 | 58 | 36 | 60 | 36 |
| | (number of | | | | | | |
| | analyzed | | | | | | |
| | spectra) | | | | | | |

FCD: Functional capillary density;
CFV: Capillary flow velocity;
PF: Pulsatile flow;
CF: Continuous flow.
SmVO₂: microvascular oxygen saturation

### Statistics

Continuous variables are presented as mean±standard deviation and displayed as box-whisker plots with median, 25th and 75th percentiles, range and outliers. Comparing conjunctival microvascular variables between the two heart pumps, an independent samples t-test was used. Comparisons of speed categories for the CFV parameter was performed with a chi square test, p<0.05 was considered statistically significant. The statistical analysis was performed by SPSS version 26 (SPSS Inc, Chicago, Illinois, USA).

### Results

### Functional Capillary Density

At baseline, FCD of all piglets was higher in limbus (11.3±3.0) as compared with bulbus (7.1±2.8), p<0.01.

By chance, the piglets which were assigned (randomly) to CF had higher FCD at baseline in limbus as compared with piglets with PF (FIG. 5A). Both groups of piglets should, theoretically, have had the same circulatory health at baseline (time on pump=0). In any event, after three and six hours, FCD decreased in the CF piglets to levels comparable to the PF piglets, which had stable FCD throughout the study.

In the bulbus, FCD did not change with either pump from baseline to three hours, but after six hours, bulbar FCD in CF piglets increased as compared with baseline recordings and as compared with the PF piglets (FIG. 5B).

### Capillary Flow Velocity

Category 0 (no flow) and 3 (continuous flow velocity) constituted 55-87% of the measurements. Category 4 (rapid flow) was rarely observed (<4% of measurements) and category 5 (brisk flow) was never observed.

In limbal recordings the PF piglets decreased category 0 and increased category 3 during the 6 hours experiment (FIGS. 6A and 6C). The opposite CFV pattern was seen in CF piglets. (FIGS. 6B and 6D).

In bulbus, piglets with PF had higher velocities at baseline as compared with CF piglets (FIG. 6E-H). During the experiment, bulbar category 3 decreased and category 0 increased independent of pump.

### Diffuse Reflectance Spectroscopy

All piglets maintained stable arterial oxygen saturation ≥99% throughout the experiment. The positioning of the DRS probe was not sufficiently precise to discriminate between limbal and bulbar recordings. SmvO₂ results pooled together decreased over time for both pumps. Since SaO₂ values were stable during the experiment, oxygen extraction (SaO₂-SmvO₂) increased after three hours and increased further after six hours (FIG. 7). There was no difference between the two groups at any time.

### Discussion

The technologies, data acquisition protocol and analyzing platform used in this study has sufficient sensitivity to characterize conjunctival microvascular function in piglets. The results show differences for all measured parameters (FCD, CFV and SmvO₂) during the 6 hours experiment, and also differences between CFV and FCD results from the piglets having slowly deteriorating circulatory health (piglets on PF pumps) versus piglets having more rapidly deteriorating circulatory health (piglets on CF pumps).

Oxygen delivery from capillaries is essential for life of human cells, and is dependent on an interaction between several factors. A complex set of physiological mechanisms (reflexes) regulate microvascular hemodynamics to optimize oxygen delivery with the least use of energy. In the conjunctiva, the critical function of this regulatory system is to maintain delivery of O₂ to limbal stem cells.

Flow and flow velocities in individual capillaries are regulated like in other blood vessels by perfusion pressure (PP) and vascular resistance (VR): CFV=PP/VR. During this experiment arterial and venous pressures were stabilized by infusions of fluids (i.e. a stable PP was maintained).

In the limbus, CFV increased in the PF piglets over time with increase in percentage in category 3 and reduction in category 0 (FIGS. 6A and 6C). The opposite trends were observed for the CF piglets (FIGS. 6B and 6D).

The limbus is the conjunctival region with a high metabolic rate secondary to production of corneal cells from large number of stem cells. The bulbar conjunctiva has in contrast few stem cells and a low metabolic rate, and microvascular blood flow has more of a transport function of blood for O₂ delivery to limbus. The higher O₂ delivery in limbus is reflected in higher FCD in limbal recordings as compared with bulbar recordings (FIG. 5). The number of visible capillaries at baseline in limbus was higher in the piglets having more rapidly deteriorating circulatory health (piglets on CF pumps) as compared with the piglets having slowly deteriorating circulatory health (piglets on PF pumps) (FIG. 5), but no changes between the two groups were seen in limbus for the next six hours on pump. In the bulbus the two groups had also similar FCD until an increase in CF piglets was seen after six hours (FIG. 5B). This recruitment of capillaries was coexisting with the mentioned reduction in CFV in CF piglets (FIGS. 6F and 6H), and can be regarded as a compensatory mechanism in CF piglets to maintain the O₂ transport capacity for blood to limbus.

In FIG. 7, all DRS data are pooled and shown irrespective of whether recordings were from limbus or bulbus. Because arterial oxygen saturation was maintained at levels >99% throughout the study, and microvascular oxygen saturation (SmvO₂) decreased for both pumps during the 6 hour study, the microvascular oxygen extraction increased with time on CPB. The increased extraction may be a compensatory mechanism to counteract the reduction in CFV with time seen in bulbus in CF piglets (FIG. 6H).

In a study in goats with an implanted total artificial heart, Baba et al. (American Society for Artificial Internal Organs, 1992; 2004; 50(4):321-7) described bulbar conjunctival microcirculation using in vivo microscopy. The implanted pump could deliver PF or CF, and capillary density and flow velocities were better preserved with PF. Hence, piglets on a PF pump were used as a model of subjects having slowly deteriorating circulatory health (i.e. having somewhat poorer circulatory health after 6 hours on CF pump), while piglets on a CF pump were used as a model of subjects with more rapidly deteriorating circulatory health (i.e. having greatly poorer circulatory health after 6 hours on CF pump). Our results showed that striking differences in CFV and FCD developed between 3 and 6 hours in the piglets on the CF pump. Results from conjunctival examinations may be used as an indicator for cerebral microvascular function.

### Example 3—Study Assessing Conjunctival Microcirculatory Measurements from the Limbus and Bulbar Conjunctiva in Human Subjects

### Material and Methods

### Study Population and Design

This cross sectional study enrolled 20 evenly gender-distributed self-reported healthy volunteers recruited amongst hospital staff and their acquaintances. Inclusion criteria were refractive error less than +/−5 spherical diopters and age 18 to 65 years. Exclusion criteria were smoking, systemic or ocular disease and previous ocular surgery.

Each participant provided a signed written informed consent in accordance with the Declaration of Helsinki human study guidelines. The study was conducted at the Department of Ophthalmology, Oslo University Hospital, following approved application to the Regional Comity for Medical and Health Ethics (REK: 2017/1352-1).

### Clinical Evaluation

Volunteers reported gender, age, weight and height in a questionnaire prior to measuring blood pressure (Dash 3000, GE healthcare, Chicago, Illinois, USA) and pulse oximetry-assessed arterial oxygen saturation (Nellcor, Medtronic, Dublin, Ireland). Intraocular pressure (iCare TA01i, iCare, Vantaa, Finland) and visual acuity were assessed in a room with standardized lighting conditions using an Early Treatment of Diabetic Retinopathy Study chart (Clear Chart 4P, Reichert, Inc., Depew, New York, USA) before one drop of oxybuprocaine (Oxibuprokain minims, 4 mg/ml, Bausch Health Ireland Ltd., Dublin, Ireland) was applied to the left conjunctival sac. At the volunteer's request, additional drops were added. Seated in a slit lamp unit, a detailed examination of the anterior and posterior segment of the eye was conducted before microvascular assessments commenced. Best corrected visual acuity of every volunteer was at least 20/20.

### Microvascular Assessments

We used the ODIN concept for repetitive ocular surface measurements of the left eye of each participant, repositioning the equipment between each recording to capture different measuring volumes (˜0.1 mm³). During analysis, custom-made software (Eyesoft 2.0, ODI Medical, Oslo, Norway) was used to quantify FCD, CFV and SmvO₂.

### Computer Assisted Video Microscopy

A slitlamp-fixated computer assisted digital microscope (CAVM) (Capillaroscope, Inspectis, Solna, Sweden) with a 200× magnification lens, 1920×1080 pixels image resolution, field of view of 1.6 mm×0.9 mm and frame rate of 30 frames per second was used to obtain films of 15 seconds duration. Five films from limbus and bulbar conjunctiva were recorded, stored and analyzed on a computer (HP Spectre x360, HP Inc, Paola Alta, California, USA). During analysis, the software created tilted (limbus) or horizontal (bulbar conjunctiva) gridlines (of which five were counted), separated by 100 microns, on top of a running film (FIG. 8). Capillaries were identified as visible blood vessels (diameter <20 μm) crossing the lines. At each crossing, capillary flow velocity (CFV) was scored according to a 6-category scale (ranging from category 0 (no flow) to category 5 (brisk flow) (the categories 0 to 5 are the same categories 0 to 5 defined in Table 1)).

FIG. 8 shows frames from limbus (A) and bulbar conjunctiva (B) during analysis.

One randomly chosen bulbar conjunctival film from each participant (n=20) was reanalyzed by the primal investigator (observer 1) and an experienced independent examiner (observer 2) to conduct inter- and intra-reliability tests.

### Diffuse Reflectance Spectroscopy

The spectrometer (AvaSpec-2048-2, Avantes, Apeldoorn, The Netherlands) was connected to a halogen light source (AvaLight-HAL, Avantes) and a measuring probe with six emitting and one receiving 400 μm fibers. For each session, calibration was conducted holding the probe against a white polytetrafluoretylene tile (WS-2, Avantes) prior to twelve spectral measurements on the tissue. During analysis, an algorithm calculated SmvO₂.

### Measuring Procedure

An external fixation target was presented to minimize eye movement during recordings. A sterile blunt insertor (23 gauge/0.6 mm, D.O.R.C. Dutch Ophthalmic Research Center (International) B.V. Zuidland, The Netherlands) was used to imprint a mark four mm from limbus in the upper temporal quadrant. Bulbar DRS spectra and CAVM recordings were recorded prior to limbal measurements.

### Statistical Analysis

Results are presented as mean±SD or median and range. A paired samples T-test was applied to FCD and SmvO₂ data. To compare CFV-results from limbus and bulbus, a dependent samples t-test was used. The significance level was set to 5%. Intra- and inter-observer reliability were determined by the intraclass correlation coefficient (ICC) and interpreted by guidelines described by Koo and Li (Journal of Chiropractic Medicine, Volume 15, Issue 2, 2016, Pages 155-163) Statistical analyses were performed in SPSS Version 26 (SPSS Inc., Chicago, Illinois, USA).

### Results

Data were collected from 20 participants (Table 4), three of which expressed modest discomfort during limbal recordings. A summary of analyzed data is given in Table 5.

TABLE 4
Baseline parameters of study participants.

| | Mean (SD) | Median (range) |
|---|---|---|
| Number of participants (♀/♂) | 20 (10/10) | 20 (10/10) |
| Age (years) ((mean (SD)) or | 38 (8) | 38 (23-51) |
| (median (range)) | | |
| Systolic blood pressure (mmHg) | 117 (9.9) | 115 (100-140) |
| Diastolic blood pressure (mmHg) | 71 (9.6) | 70 (50-85) |
| Pulse oximetry (%) | 98 (1.1) | 98 (96-100) |
| Heart rate (beats per minute) | 63 (11.9) | 60 (49-89) |
| Intraocular pressure os (mmHg) | 15 (2.9) | 15 (10-22) |

TABLE 5
Overview of analyzed data.

| | | Bulbar |
|---|---|---|
| Parameter | Limbus | conjunctiva |
| FCD¹ | 2855 | 3738 |
| CFV¹ | 1474 | 2238 |
| SmvO2² | 228 | 221 |

Abbreviations: FCD: Functional capillary density, CFV: Capillary flow velocity, SmvO₂: ¹Number of assessed capillaries ²Number of analyzed spectra Microvascular oxygen saturation.

### Functional Capillary Density

Functional capillary density at limbus (11.2 (1.8) c/mm) was larger than bulbar conjunctiva (5.2 (1.2) c/mm), p<0.01. Intra-observer reliability between the first and second analyses by the primary investigator was good (0.78) whereas inter-observer reliability between two independent examiners was moderate (0.72).

FIG. 9 shows Ocular surface functional capillary density measured in the limbus and bulbar conjunctiva. Each dot represents the mean of valid recordings from one volunteer. Horizontal lines represent the mean of each data set.

### Capillary Flow Velocity

At least 80% of scored limbal and bulbar capillaries were category 3 and there was no difference in flow patterns comparing anatomical locations, p 0.68

FIG. 10 shows ocular surface capillary flow velocity. Each dot represents the mean of valid recordings from one volunteer. Horizontal lines represent the mean of each data set.

### Microvascular Oxygen Saturation

Microvascular oxygen saturation was lower at the limbus (77% (8)) at limbus as compared to bulbar conjunctiva (89 (6) %), p<0.01.

FIG. 11 shows ocular surface microvascular oxygen saturation. Each dot represents the mean value within one individual's recordings. Horizontal lines represent the mean of each data set.

### CONCLUSION

The technology used in this study can be used to assess conjunctival microcirculation in humans without harm to the examined individual. The technology and analyzing platform had sufficient precision to measure CFV and FCD (using microscopy) and SmvO₂ (using DRS) in both the limbus and the bulbar conjunctiva.

The technology and analyzing platform had sufficient precision to show that the limbus of the eye had a higher metabolic activity as compared with the bulbar conjunctiva. This result confirms the findings of Examples 1 and 2. The technology is a new tool for studying eye diseases (e.g. glaucoma, macular degeneration) and monitoring progression of diseases (e.g. diabetes mellitus). The tool may be used for studying circulatory disturbances in the brain (e.g. ischemic or hemorrhagic stroke) and for assessing the severity of systemic circulatory failure (e.g. acute heart failure treated with ECMO-extra corporal membrane oxygenation).

1. A method of identifying or monitoring circulatory failure in a subject, or making a prognosis for a subject, or providing clinically relevant information about a subject, or monitoring the efficacy of treatment in a subject,
which method comprises
assessing the microcirculation in the limbus and optionally also the bulbar conjunctiva of the subject, said method comprising assessing the following parameter(s) in the subject's limbus and optionally also in their bulbar conjunctiva:
(i) functional capillary density (FCD); and/or
(ii) capillary flow velocity (CFV);
wherein parameters (i) and (ii) are assessed by microscopy.

2. The method of claim 1, wherein the assessing of parameter(s) (i) and/or (ii) in the limbus of the subject comprises obtaining value(s) for parameter(s) (i) and/or (ii) in the limbus of the subject.

3. The method of claim 2, wherein values for parameters (i) and (ii) in the limbus of the subject are obtained.

4. The method of claim 2 or claim 3, further comprising comparing the value obtained for parameter (i) and/or (ii) in the limbus of the subject with a healthy reference value or a value for the subject from an earlier point in time.

5. The method of any one of claims 2 to 4, wherein the following outcome or combination of outcomes is indicative of circulatory failure in said subject:
the value obtained for one of parameters (i) and (ii) in the limbus of the subject is below the corresponding healthy reference value, or value for the subject from an earlier point in time, and/or
the value obtained for the other parameter in the limbus of the subject is above the corresponding healthy reference value, or value for the subject from an earlier point in time.

6. The method of any one of claims 2 to 5, wherein the following outcome is indicative of circulatory failure in said subject:
the value obtained for parameter (ii) in the limbus of the subject is below the corresponding healthy reference value, or value for the subject from an earlier point in time.

7. The method of any preceding claim, further comprising
assessing the microcirculation in the bulbar conjunctiva of the subject, comprising assessing the following parameter(s) in the subject's bulbar conjunctiva:
(i) functional capillary density (FCD); and/or
(ii) capillary flow velocity (CFV);
wherein parameters (i) and (ii) are assessed by microscopy.

8. The method of claim 7, wherein the assessing of parameter(s) (i) and/or (ii) in the bulbar conjunctiva of the subject comprises obtaining value(s) for parameter(s) (i) and/or (ii) in the bulbar conjunctiva of the subject.

9. The method of claim 8, wherein values for parameters (i) and (ii) in the bulbar conjunctiva of the subject are obtained.

10. The method of claim 8 or claim 9, further comprising comparing the value obtained for parameter (i) and/or (ii) in the bulbar conjunctiva of the subject with a healthy reference value or a value for the subject from an earlier point in time.

11. The method of any one of claims 8 to 10, wherein the following outcome or combination of outcomes is indicative of circulatory failure in said subject:
the value obtained for one of parameters (i) and (ii) in the bulbar conjunctiva of the subject is below the corresponding healthy reference value, or value for the subject from an earlier point in time, and/or
the value obtained for the other parameter in the bulbar conjunctiva of the subject is above the corresponding healthy reference value, or value for the subject from an earlier point in time.

12. The method of any one of claims 8 to 11, wherein the following outcome or combination of outcomes is indicative of circulatory failure in said subject:
the value obtained for parameter (i) in the bulbar conjunctiva of the subject is above the corresponding healthy reference value, or value for the subject from an earlier point in time, and/or
the value obtained for parameter (ii) in the bulbar conjunctiva of the subject is below the corresponding healthy reference value, or value for the subject from an earlier point in time.

13. The method of any preceding claim, further comprising making a diagnosis of circulatory failure in said subject when circulatory failure is indicated.

14. The method of any preceding claim, further comprising altering, ceasing or continuing treatment of said subject.

15. The method of any preceding claim, wherein the microscope uses unpolarised and/or polychromatic light, preferably white unpolarised light.

16. The method of any preceding claim, wherein the microscope is a video microscope, preferably a computer assisted video microscope (CAVM).

17. The method of any preceding claim, wherein the assessment is performed on data previously acquired from the subject and the subject is not still undergoing monitoring.

18. The method of any preceding claim, wherein one or more of the following parameters are also assessed:
(a) heterogeneity of the FCD;
(b) heterogeneity of CFV;
(c) oxygen saturation of microvascular erythrocytes (SmvO₂); and
(d) heterogeneity of SmvO₂;
preferably wherein parameters (a) and (b) are assessed by microscopy and parameters (c) and (d) are assessed by diffuse reflectance spectroscopy (DRS).

19. The method of claim 18, wherein 2 or more, more preferably 3 or 4 of parameters (a) to (d) are assessed, most preferably in both the limbus and bulbar conjunctiva.

20. The method of any preceding claim, wherein the subject is:
(i) being considered for or undergoing intensive care therapy, such as extra-corporeal membrane oxygenation (ECMO) and/or extra-corporeal life support treatment (ECLS); or
(ii) suffering from pre-eclampsia; or
(iii) suffering from sepsis; or
(iv) suffering from chronic or acute heart failure; or
(v) asphyxiated; or
(vi) suffering from acute or chronic respiratory failure; or
(vii) the recipient of an organ transplant.

21. The method of any preceding claim, wherein the subject is suffering from:
(i) An eye condition, preferably selected from glaucoma, wet age-related macular degeneration (wet AMD), pterygium, eye cancer, local inflammation, scleritis/episcleritis, stem cell failure, corneal transplant failure, keratoconus, ocular ischemic syndrome, Anterior Ischemic Optic Neuropathy (AION) or complication following cosmetic procedures performed on the eye or eye lids; or
(ii) A neurological condition, for example a circulatory disturbance of the brain such as ischemic or hemorrhagic stroke, multiple sclerosis, meningitis or increased intracranial pressure; or
(iii) Hypovolemic shock, diabetes with microangiopathy or prematurity.

22. The method of claim 21, wherein the eye cancer is uveal melanoma, conjunctival melanoma or basal cell cancer of the eyelid.

23. The method of claim 21 or claim 22, wherein the local inflammation of the eye is vasculitis, uveitis or due to allergy.

24. The method of any one of claims 21 to 23, wherein the stem cell failure in the eye is Fuchs dystrophia.

25. The method of any one of claims 21 to 24, wherein the AION is arteritic AION or non-arteritic AION, and preferably is due to retinal artery embolus, retinal vein occlusion, chemical burns, or stroke.

26. The method of any preceding claim, wherein the subject has conjunctival pigmentation.

27. Apparatus for assessing microcirculation in the limbus of a subject comprising a microscope, optionally a spectrometer, and a computer, whereby the computer is arranged to receive image(s) of the microcirculation obtained using the microscope and optionally data relating to SmvO₂ from the spectrometer and, optionally, to process the image(s) and data to identify and/or determine characteristics/parameters associated with pathology, wherein the image(s) and data relate to the following parameters:
(i) functional capillary density (FCD); and/or
(ii) capillary flow velocity; and optionally one or more of:
(a) heterogeneity of the FCD;
(b) heterogeneity of CFV;
(c) oxygen saturation of microvascular erythrocytes (SmvO₂); and
(d) heterogeneity of SmvO₂.

28. The apparatus of claim 27, wherein the apparatus is also for assessing microcirculation in another conjunctival region of the subject, preferably the bulbar conjunctiva.

29. Apparatus for assessing microcirculation in the limbus of a subject comprising a computer arranged to receive image(s) of the microcirculation of a subject's limbus obtained using a microscope and optionally data relating to SmvO₂ from a spectrometer and to process the image(s) and data to identify and/or determine characteristics/parameters associated with pathology, wherein the image(s) and data relate to the following parameters:
(i) functional capillary density (FCD); and/or
(ii) capillary flow velocity; and optionally one or more of:
(a) heterogeneity of the FCD;
(b) heterogeneity of CFV;
(c) oxygen saturation of microvascular erythrocytes (SmvO₂); and
(d) heterogeneity of SmvO₂.

30. The apparatus of claims 27 to 29, further comprising a means for outputting values corresponding to the characteristics/parameters and/or a value based on them in combination, preferably a weighted sum or average.

31. Software comprising instructions to cause a computer to carry out the image processing and/or output steps defined in any one of claims 27 to 30.

32. A method of identifying or monitoring circulatory failure in a subject, or making a prognosis for a subject, or providing clinically relevant information about a subject, or monitoring the efficacy of treatment in a subject,
which method comprises
processing, implemented by a computer, of image(s) of the microcirculation in the limbus and optionally also the bulbar conjunctiva of the subject previously acquired using a microscope, to obtain value(s) for the following parameter(s) in the subject's limbus and optionally also in their bulbar conjunctiva:
(i) functional capillary density (FCD); and/or
(ii) capillary flow velocity (CFV).

33. The method of claim 32, further comprising
processing, implemented by a computer, of image(s) of the microcirculation in the bulbar conjunctiva of the subject, to obtain value(s) for the following parameter(s) in the subject's bulbar conjunctiva:
(i) functional capillary density (FCD); and/or
(ii) capillary flow velocity (CFV);
wherein parameters (i) and (ii) are assessed by microscopy.

34. The method of claim 32 or claim 33, wherein value(s) for one or more of the following parameters are also obtained:
(a) heterogeneity of the FCD;
(b) heterogeneity of CFV;
(c) oxygen saturation of microvascular erythrocytes (SmvO₂); and
(d) heterogeneity of SmvO₂;
preferably wherein the value(s) of parameters (a) and (b) are generated using the image(s) and the value(s) of parameters (c) and (d) are generated using data previously acquired by diffuse reflectance spectroscopy (DRS).

35. The method of claim 34, wherein value(s) for 2 or more, more preferably 3 or 4 of parameters (a) to (d) are obtained, most preferably in both the limbus and bulbar conjunctiva.

36. The method of any one of claims 32 to 35, comprising the features of any one of claims 2 to 6, 8 to 17, and 20 to 26.
